(19) United States
(12) Patent Application Publication
(10) Pub. No.: US 2023/0323385 A1
(43) Pub. Date: Oct. 12, 2023

(54) PLANTS WITH IMPROVED NEMATODE RESISTANCE

(71) Applicants: Syngenta Crop Protection AG, Basel (CH); INSTITUT NATIONAL DE RECHERCHE POUR L'AGRICULTURE, L'ALIMENTATION ET L'ENVIRONNEMENT, Paris (FR); UNIVERSITÉ COTE D'AZUR, Nice (FR); CENTRE NATIONAL DE LA RECHERCHE SCIENTIFIQUE, Paris (FR)

(72) Inventors: Michaël Quentin, Sophia Antipolis (FR); Bruno Favery, Sophia Antipolis (FR); Gregori Bonnet, Sarrians (FR); Joffrey Mejias, Sophia Antipolis (FR); Nhat-My Truong, Sophia Antipolis (FR); Pierre Abad, Sophia Antipolis (FR)

(73) Assignees: SYNGENTA CROP PROTECTION AG, Basel (CH); INSTITUT NATIONAL DE RECHERCHE POUR L'AGRICULTURE, L'ALIMENTATION ET L'ENVIRONNEMENT, Paris (FR); UNIVERSITÉ COTE D'AZUR, Nice (FR); CENTRE NATIONAL DE LA RECHERCHE SCIENTIFIQUE, Paris (FR)

(21) Appl. No.: 17/995,119

(22) PCT Filed: Mar. 29, 2021
(86) PCT No.: PCT/EP2021/058173

§ 371 Date: Sep. 30, 2022

(30) Foreign Application Priority Data
Apr. 3, 2020 (EP) 20167882.8

(51) Int. Cl. C12N 15/82; A01H 6/82; C12N 15/10; C12Q 1/6895

(52) U.S. Cl. CPC C12N 15/8285; A01H 6/825; C12N 15/102; C12Q 1/6895; C12Q 2600/13; C12Q 2600/156

(57) ABSTRACT

The present invention relates to novel plants displaying an improved resistance to nematodes. The present invention also relates to seeds and parts of said plants. The present invention further relates to methods of making and using such seeds and plants. The present invention also relates to a novel SmD1 allele, which results in a modified SmD1 protein associated with such improved resistance to nematodes.

### FIELD OF THE INVENTION

The present invention relates to novel plants displaying an improved resistance to nematodes. The present invention also relates to seeds and parts of said plants. The present invention further relates to methods of making and using such seeds and plants. The present invention also relates to a novel SmD1 allele, which results in a modified SmD1 protein associated with such improved resistance to nematodes.

### BACKGROUND OF THE INVENTION

Sedentary endoparasitic nematodes, such as root-knot nematodes (RKN; Meloidogyne spp.) and cyst nematodes (CN; Heterodera spp. and Globodera spp.) cause considerable damage to many agricultural crops. Nematodes spend most of their life cycle in plant roots, in which they induce the formation of multinucleate hypertrophied feeding cells, called giant cells and syncytia, respectively. These giant cells, surrounded by small dividing cells and forming a new organ within the root known as a root knot or gall, then act as metabolic sinks from which the nematode feeds nutrients throughout its life. This leads to severe defects in the functionality of the plant root system, drastically reducing the efficiency of plant nutrient uptake and ultimately affecting yield (Singh et al., 2013; Mejias et al., 2019).

Nematode control to prevent yield loss usually relies on crop management and rotation, use of nematicides and plant genetics. However, many nematicides solutions are withdrawn from the marketplace. Furthermore, their use has been drastically reduced to address human health, food safety concerns (e.g. regarding residues on crop harvests) and environmental sustainability (e.g. preserving soil life). Furthermore, some nematodes can overcome the very few existing solutions based on plant genetics. For instance, many Meloidogyne species (e.g. M. enterolobii, incognita, arenaria and javanica) can overcome the resistance of tomato and pepper genotypes carrying the Mi-1.2 and N resistance genes widely used for nematode management (Kiewnick et al., 2009).

Consequently, there is a need for alternative solutions to further improve nematode control in plants, especially in tomato plants.

### SUMMARY OF THE INVENTION

The present invention addresses the need for providing novel plants exhibiting an increased resistance to nematodes, especially to nematodes of the genus Meloidogyne. In a first embodiment, the invention provides a plant comprising a SmD1 allele encoding a SmD1 protein having at least 90% amino acid sequence identity with SEQ ID NO: 1, wherein said SmD1 protein comprises a missense mutation resulting in a modified SmD1 protein conferring an improved nematode resistance.

In a further embodiment, said modified SmD1 protein comprises a missense mutation at a position corresponding to any one of amino acid positions 1 to 108 of SEQ ID NO: 1.

In a further embodiment, said modified SmD1 protein comprises a missense mutation at a position corresponding to amino acid position 14 of SEQ ID NO: 1.

In a further embodiment, said modified SmD1 protein comprises a threonine to isoleucine substitution at a position corresponding to amino acid position 14 of SEQ ID NO: 1.

In a further embodiment, the invention provides a plant according to any of the preceding embodiments, wherein said plant is selected from the list comprising tomato, tobacco, pepper, squash, watermelon, melon, cucumber and soy.

In a further embodiment, the invention provides a plant according to any of the preceding embodiments, wherein said plant is an inbred, a dihaploid or a hybrid plant.

In a further embodiment, the invention provides a plant according to any of the preceding embodiments, wherein said plant is a rootstock.

In a further embodiment, the invention provides a plant according to any of the preceding embodiments, wherein said plant comprises two copies of said SmD1 allele.

In a further embodiment, said modified SmD1 protein confers an improved resistance against the nematodes of the genera Meloidogyne, preferably Meloidogyne incognita, Meloidogyne arenaria, Meloidogyne hapla, Meloidogyne enterolobii and Meloidogyne javanica.

In a further embodiment, the invention provides a plant according to any of the preceding embodiments, wherein said plant is Solanum lycopersicum.

In a further embodiment, said modified SmD1 protein has an amino acid sequence of SEQ ID NO: 2.

In a further embodiment, said SmD1 allele is obtainable from Solanum lycopersicum line 19TEP250122, deposited with NCIMB on 29 Nov. 2019 under NCIMB Accession No. 43529.

In a further embodiment, the invention provides a plant part according to any of the preceding embodiments, wherein said plant part comprises said SmD1 allele.

In a further embodiment, the invention provides a seed that produces the plant or plant part of any of the preceding embodiments.

In a further embodiment, the invention provides a method of improving nematode resistance in a plant, comprising the steps of
a) Obtaining a population of mutant plants;
b) Selecting a mutant plant comprising a modified SmD1 allele encoding a SmD1 protein with a missense mutation in its amino acid sequence.

The use of a SmD1 allele resulting in a modified SmD1 protein has been shown to result in an increased tolerance to nematodes. It was demonstrated that a missense mutation allows to maintain the required activity of the modified SmD1 protein in planta while preventing said SmD1 protein from being recognized by nematode effectors, thereby improving the ability of the plant to cope with the pest. This invention therefore has the potential to be used in future breeding programs for improving plant resistance against nematode pests.

### BRIEF DESCRIPTION OF THE DRAWINGS

FIG. 1: Sequence alignments and percent identity matrix of the SmD1 amino acid sequences of the invention, encoded by Arabidopsis thaliana (AT4G02840.1 (SEQ ID NO: 4) and AT3G07590.1 (SEQ ID NO: 5)), Nicotiana benthamiana (NbS00005390g0012.1 (SEQ ID NO: 6), NbS00006569g0006.1 (SEQ ID NO: 7) and NbS00054309g0007.1 (SEQ ID NO: 8)) and Solanum lycopersicum (Solyc09g064660.2.1 (SEQ ID NO: 1) and Solyc06g084310.2.1 (SEQ ID NO: 3)), and the orthologous sequences from Glycine max (Glyma.02G096000.1 (SEQ ID NO: 9)), Capsicum annuum (CA06g26820 (SEQ ID NO: 10) and Capana06g000068 (SEQ ID NO: 11)), Cucurbita moschata (CmoCh02G018520.T1 (SEQ ID NO: 12)), Cucumis melo (MELO3C018220.2.1 (SEQ ID NO: 13)), Cucumis sativus (Cs.gyl4.3.1.022189.T1 (SEQ ID NO: 14)), Citrillus lanatus (Cla023415_T (SEQ ID NO: 15)), Solanum habrochaites (Sh.LY101.2.1.003421.T1 (SEQ ID NO: 16)) and Solanum pennellii (Sopen09g026350.1 (SEQ ID NO: 17)) SmD1 alleles. Sequence alignments and percent identity matrix were calculated using the software Clustal Omega (Sievers et al., 2011).

FIG. 2: Assessment of the level of susceptibility to nematodes in Arabidopsis (A), Nicotiana benthamiana (B) and tomato (C) plants with impaired expression of SmD1 genes versus respective control plants. (D) Assessment of the plant root system in SmD1 silenced tomato plants. (A) Forty plants were used for each genotype and the statistical analysis of the results was done with Student's t-test (P<0.05). (B) Twelve plants were used for each treatment and the statistical analysis of the results was done with Mann-Whitney test (α=5%). (C) Between eighteen and twenty plants were used for each genotype respectively and the statistical analysis of the results was done with Mann-Whitney test (α=5%).

FIG. 3: Assessment of the plant root system (A) and of the level of susceptibility to nematodes (B) of tomato plants with a missense mutation in their SmD1b gene. The statistical analysis of the results was done with a Mann-Whitney test (α=1%).

### BRIEF DESCRIPTION OF THE SEQUENCES.

SEQ ID NO:1: Amino acid sequence encoded by SmD1b gene Solyc09g064660.2.1
SEQ ID NO:2: Modified amino acid sequence comprising a T14I missense mutation at position 14 of SEQ ID NO:1
SEQ ID NO:3: Amino acid sequence encoded by SmD1a gene Solyc06g084310.2.1
SEQ ID NO:4: Amino acid sequence encoded by SmD1b gene AT4G02840.1
SEQ ID NO:5: Amino acid sequence encoded by SmD1a gene AT3G07590.1
SEQ ID NO:6: Amino acid sequence encoded by SmD1 gene NbS00005390g0012.1
SEQ ID NO:7: Amino acid sequence encoded by SmD1 gene NbS00006569g0006.1
SEQ ID NO:8: Amino acid sequence encoded by SmD1 gene NbS00054309g0007.1
SEQ ID NO:9: Amino acid sequence encoded by SmD1 gene Glyma.02G096000.1
SEQ ID NO:10: Amino acid sequence encoded by SmD1 gene CA06g26820
SEQ ID NO:11: Amino acid sequence encoded by SmD1 gene Capana06g000068
SEQ ID NO:12: Amino acid sequence encoded by SmD1 gene CmoCh02G018520.T1
SEQ ID NO:13: Amino acid sequence encoded by SmD1 gene MELO3C018220.2.1
SEQ ID NO:14: Amino acid sequence encoded by SmD1 gene Cs.gy14.3.1.022189.T1
SEQ ID NO:15: Amino acid sequence encoded by SmD1 gene Cla023415_T
SEQ ID NO:16: Amino acid sequence encoded by SmD1 gene Sh.LY101.2.1.003421.T1
SEQ ID NO:17: Amino acid sequence encoded by SmD1 gene Sopen09g026350.1
SEQ ID NO:18: Nucleic acid sequence encoding SEQ ID NO:1
SEQ ID NO:19: Nucleic acid sequence encoding SEQ ID NO:2
SEQ ID NO:20: Genomic sequence of Solyc09g064660.2.1 SmD1b gene
SEQ ID NO: 21: Genomic sequence of modified Solyc09g064660.2.1 SmD1b gene
SEQ ID NO:22/23: Primer pair to amplify Solyc09g064660.2.1 gene region
SEQ ID NO:24: Genomic sequence encoding SEQ ID NO:3
SEQ ID NO:25: Genomic sequence encoding SEQ ID NO:4
SEQ ID NO:26: Genomic sequence encoding SEQ ID NO:5
SEQ ID NO:27: Genomic sequence encoding SEQ ID NO:9
SEQ ID NO:28: Genomic sequence encoding SEQ ID NO:10
SEQ ID NO:29: Genomic sequence encoding SEQ ID NO:11
SEQ ID NO:30: Genomic sequence encoding SEQ ID NO:12
SEQ ID NO:31: Genomic sequence encoding SEQ ID NO:13
SEQ ID NO:32: Genomic sequence encoding SEQ ID NO:14
SEQ ID NO:33: Genomic sequence encoding SEQ ID NO:15
SEQ ID NO:34: Genomic sequence encoding SEQ ID NO:16
SEQ ID NO:35: Genomic sequence encoding SEQ ID NO:17

### DETAILED DESCRIPTION OF THE INVENTION

### Definitions

The technical terms and expressions used within the scope of this application are generally to be given the meaning commonly applied to them in the pertinent art of plant breeding and cultivation if not otherwise indicated herein below.

As used in this specification and the appended claims, the singular forms “a”, “an”, and “the” include plural referents unless the context clearly dictates otherwise. Thus, for example, reference to “a plant” includes one or more plants, and reference to “a cell” includes mixtures of cells, tissues, and the like.

As used herein, the term “about” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

A “cultivated” plant is understood within the scope of the invention to refer to a plant that is no longer in the natural state but has been developed and domesticated by human care for agricultural use and/or human consumption and excludes wild accessions. As a matter of example, in embodiments, the “cultivated plant” is a hybrid plant. Alternatively, or additionally, a “cultivated tomato” plant according to the present invention is capable of growing yellow, orange or red fruits. Alternatively, or additionally, the cultivated tomato plant is a Solanum lycopersicum plant.

An “allele” is understood within the scope of the invention to refer to alternative or variant forms of various genetic units identical or associated with different forms of a gene, which are alternative in inheritance because they are situated at the same locus in homologous chromosomes. Such alternative or variant forms may be the result of single nucleotide polymorphisms, insertions, inversions, translocations or deletions, or the consequence of gene regulation caused by, for example, by chemical or structural modification, transcription regulation or post-translational modification/regulation. In a diploid cell or organism, the two alleles of a given gene or genetic element typically occupy corresponding loci on a pair of homologous chromosomes. In the context of the present invention, an alternative or variant allele of a SmD1 gene encodes a modified SmD1 protein comprising a missense mutation associated with an improved nematode resistance phenotype. The alternative or variant allele of a SmD1 gene is defined relatively to the wild type SmD1 gene. For example, wild type SmD1b gene sequence of SED ID NO: 20 encodes wild type SmD1b protein of SEQ ID NO: 1. Correspondingly, variant SmD1b allele of SEQ ID NO: 21 encodes modified SmD1b protein of SEQ ID NO: 2.

Relatively speaking, the term “improved nematode resistance” is herein understood to mean that a plant according to the present invention, e.g. comprising a SmD1 allele encoding a SmD1 protein having at least 90% amino acid sequence identity with SEQ ID NO: 1, wherein said SmD1 protein comprises a missense mutation, exhibits an increased nematode resistance when compared with a plant lacking said allele. A plant with an “improved nematode resistance” is understood within the scope of the invention to mean plant which has a statistically significant increased nematode resistance compared to a control plant (for example exhibits a significant decrease in the number of egg masses, as described in the Example section), using Mann-Whitney test (α=1, 2.5 or 5%) or Student's test (P<0.05).

The term “intermediate resistance” in the context of nematode resistance refers to a plant comprising an allele according to the present invention and showing a statistical significant difference in term of number of root knots and/or number of egg masses when compared with a susceptible control plant harbouring a wild type corresponding allele (Tomato Reference Genome—HEINZ).

A “control plant” within the scope of the invention can be a plant that has the same genetic background as the cultivated plant of the present invention wherein the control plant does not have the alleles of the present invention linked to improved nematode resistance. A control plant can be a plant belonging to the same plant variety and does not comprise the alleles of the invention. The control plant is grown for the same length of time and under the same conditions as the cultivated plant of the present invention. Plant variety is herein understood according to definition of UPOV. Thus, a control plant may be a near-isogenic line, an inbred line or a hybrid provided that they have the same genetic background as the plant of the present invention except the control plant does not have any of the alleles of the present invention linked to improved nematode resistance. In a preferred embodiment, the “control plant” is a “control tomato plant”.

The term “trait” refers to a characteristic or a phenotype. In the context of the present invention, a nematode resistance trait is an improved nematode resistance trait. A trait may be inherited in a dominant or recessive manner, or in a partial or incomplete-dominant manner. A trait may be monogenic or polygenic or may result from the interaction of one or more genes with the environment. A plant can be homozygous or heterozygous for the trait.

The terms “hybrid”, “hybrid plant”, and “hybrid progeny” refer to an individual produced from genetically different parents (e.g. a genetically heterozygous or mostly heterozygous individual).

The term “inbred line” refers to a genetically homozygous or nearly homozygous population. An inbred line, for example, can be derived through several cycles of brother/sister breeding or of selfing or in dihaploid production.

The term “dihaploid line” refers to stable inbred lines issued from anther culture. Some pollen grains (haploid) cultivated on specific medium and circumstances can develop plantlets containing n chromosomes. These plantlets are then “doubled” and contain 2n chromosomes. The progeny of these plantlets is named “dihaploid” and are essentially no longer segregating (stable).

The term “cultivar” or “variety” refers to a horticultural derived variety, as distinguished from a naturally occurring variety. In some embodiments of the present invention the cultivars or varieties are commercially valuable.

The term “rootstock” refers to a plant used as a receptacle for a scion plant. Typically, the rootstock plant and the scion plant are of different genotypes. In embodiments, plants according to the present invention are used as rootstock plants.

The term “genetically fixed” refers to a genetic element which has been stably incorporated into the genome of a plant that normally does not contain the genetic element. When genetically fixed, the genetic element can be transmitted in an easy and predictable manner to other plants by sexual crosses.

The term “plant” or “plant part’ refers hereinafter to a plant part, organ or tissue obtainable from a (e.g. tomato) plant according to the invention, including but not limiting to leaves, stems, roots, flowers or flower parts, fruits, shoots, gametophytes, sporophytes, pollen, anthers, microspores, egg cells, zygotes, embryos, meristematic regions, callus tissue, seeds, cuttings, cell or tissue cultures or any other part or product of the plant which still exhibits the nematode resistance trait according to the invention, particularly when grown into a plant that produces fruits.

A “plant” is any plant at any stage of development.

A “plant seed” is a seed which grows into a plant according to any of the embodiments.

A “plant cell” is a structural and physiological unit of a plant, comprising a protoplast and a cell wall. The plant cell may be in form of an isolated single cell or a cultured cell, or as a part of higher organized unit such as, for example, plant tissue, a plant organ, or a whole plant.

“Plant cell culture” means cultures of plant units such as, for example, protoplasts, cell culture cells, cells in plant tissues, pollen, pollen tubes, ovules, embryo sacs, zygotes and embryos at various stages of development.

A “plant organ” is a distinct and visibly structured and differentiated part of a plant such as a root, stem, leaf, flower bud, or embryo.

“Plant tissue” as used herein means a group of plant cells organized into a structural and functional unit. Any tissue of a plant in planta or in culture is included. This term includes, but is not limited to, whole plants, plant organs, plant seeds, tissue culture and any groups of plant cells organized into structural and/or functional units. The use of this term in conjunction with, or in the absence of, any specific type of plant tissue as listed above or otherwise embraced by this definition is not intended to be exclusive of any other type of plant tissue.

As used herein, the term “breeding”, and grammatical variants thereof, refer to any process that generates a progeny individual. Breeding can be sexual or asexual, or any combination thereof. Exemplary non-limiting types of breeding include crossings, selfing, doubled haploid derivative generation, and combinations thereof.

As used herein, the phrase “established breeding population” refers to a collection of potential breeding partners produced by and/or used as parents in a breeding program; e.g., a commercial breeding program. The members of the established breeding population are typically well-characterized genetically and/or phenotypically. For example, several phenotypic traits of interest might have been evaluated, e.g., under different environmental conditions, at multiple locations, and/or at different times. Alternatively or in addition, one or more genetic loci associated with expression of the phenotypic traits might have been identified and one or more of the members of the breeding population might have been genotyped with respect to the one or more genetic loci as well as with respect to one or more genetic markers that are associated with the one or more genetic loci.

As used herein, the phrase “diploid individual” refers to an individual that has two sets of chromosomes, typically one from each of its two parents. However, it is understood that in some embodiments a diploid individual can receive its “maternal” and “paternal” sets of chromosomes from the same single organism, such as when a plant is selfed to produce a subsequent generation of plants.

“Homozygous” is understood within the scope of the invention to refer to like alleles at one or more corresponding loci on homologous chromosomes. In the context of the invention, a plant comprising two identical copies of a particular allele at a particular locus, e.g. a SmD1 allele encoding a SmD1 protein having at least 90% amino acid sequence identity with SEQ ID NO: 1, wherein said SmD1 protein comprises a missense mutation resulting in a modified SmD1 protein conferring an improved nematode resistance, is homozygous at a corresponding locus.

“Heterozygous” is understood within the scope of the invention to refer to unlike alleles at one or more corresponding loci on homologous chromosomes. In the context of the invention, a tomato plant comprising one copy of a particular allele at a particular locus, e.g. a SmD1 allele encoding a SmD1 protein having at least 90% amino acid sequence identity with SEQ ID NO: 1, wherein said SmD1 protein comprises a missense mutation resulting in a modified SmD1 protein conferring an improved nematode resistance, is heterozygous at a corresponding locus.

A “dominant” allele is understood within the scope of the invention to refer to an allele which determines the phenotype when present in the heterozygous or homozygous state.

A “recessive” allele refers to an allele which determines the phenotype when present in the homozygous state only.

A “missense mutation” is understood to refer to a point mutation in which a single nucleotide change results in a codon that codes for a different amino acid.

“Backcrossing” is understood within the scope of the invention to refer to a process in which a hybrid progeny is repeatedly crossed back to one of the parents. Different recurrent parents may be used in subsequent backcrosses.

“Locus” is understood within the scope of the invention to refer to a region on a chromosome, which comprises a gene or any other genetic element or factor contributing to a trait.

As used herein, “marker locus” refers to a region on a chromosome, which comprises a nucleotide or a polynucleotide sequence that is present in an individual's genome and that is associated with one or more loci of interest, which may comprise a gene or any other genetic determinant or factor contributing to a trait. “Marker locus” also refers to a region on a chromosome, which comprises a polynucleotide sequence complementary to a genomic sequence, such as a sequence of a nucleic acid used as probes.

As used herein, the phrases “sexually crossed” and “sexual reproduction” in the context of the presently disclosed subject matter refers to the fusion of gametes to produce progeny (e.g., by fertilization, such as to produce seed by pollination in plants). A “sexual cross” or “cross-fertilization” is in some embodiments the fertilization of one individual by another (e.g., cross-pollination in plants). The term “selfing” refers in some embodiments to the production of seed by self-fertilization or self-pollination; i.e. pollen and ovule are from the same plant.

As used herein, the phrase “genetic marker” refers to a feature of an individual's genome (e.g., a nucleotide or a polynucleotide sequence that is present in an individual's genome) that is associated with one or more loci of interest. In some embodiments, a genetic marker is polymorphic in a population of interest, or the locus occupied by the polymorphism, depending on context. Genetic markers include, for example, single nucleotide polymorphisms (SNPs), indels (i.e., insertions/deletions), simple sequence repeats (SSRs), restriction fragment length polymorphisms (RFLPs), random amplified polymorphic DNAs (RAPDs), cleaved amplified polymorphic sequence (CAPS) markers, Diversity Arrays Technology (DArT) markers, and amplified fragment length polymorphisms (AFLPs), among many other examples. Genetic markers can, for example, be used to locate genetic loci containing alleles on a chromosome that contribute to variability of phenotypic traits. The phrase “genetic marker” can also refer to a polynucleotide sequence complementary to a genomic sequence, such as a sequence of a nucleic acid used as probes.

A “genetic marker” can be physically located in a position on a chromosome that is within or outside the genetic locus with which it is associated (i.e., is intragenic or extragenic, respectively). Stated another way, whereas genetic markers are typically employed when the location on a chromosome of the gene or of a functional mutation, e.g. within a control element outside of a gene, that corresponds to the locus of interest has not been identified and there is a non-zero rate of recombination between the genetic marker and the locus of interest, the presently disclosed subject matter can also employ genetic markers that are physically within the boundaries of a genetic locus (e.g., inside a genomic sequence that corresponds to a gene such as, but not limited to a polymorphism within an intron or an exon of a gene). In some embodiments of the presently disclosed subject matter, the one or more genetic markers comprise between one and ten markers, and in some embodiments the one or more genetic markers comprise more than ten genetic markers.

As used herein, the term “genotype” refers to the genetic constitution of a cell or organism. An individual's “genotype for a set of genetic markers” includes the specific alleles, for one or more genetic marker loci, present in the individual's haplotype. As is known in the art, a genotype can relate to a single locus or to multiple loci, whether the loci are related or unrelated and/or are linked or unlinked. In some embodiments, an individual's genotype relates to one or more genes that are related in that the one or more of the genes are involved in the expression of a phenotype of interest (e.g., a quantitative trait as defined herein). Thus, in some embodiments a genotype comprises a summary of one or more alleles present within an individual at one or more genetic loci of a quantitative trait. In some embodiments, a genotype is expressed in terms of a haplotype (defined herein below).

As used herein, the term “germplasm” refers to the totality of the genotypes of a population or other group of individuals (e.g., a species). The term “germplasm” can also refer to plant material; e.g., a group of plants that act as a repository for various alleles. The phrase “adapted germplasm” refers to plant materials of proven genetic superiority; e.g., for a given environment or geographical area, while the phrases “non-adapted germplasm,” “raw germplasm,” and “exotic germplasm” refer to plant materials of unknown or unproven genetic value; e.g., for a given environment or geographical area; as such, the phrase “non-adapted germplasm” refers in some embodiments to plant materials that are not part of an established breeding population and that do not have a known relationship to a member of the established breeding population.

As used herein, the phrase “nucleic acid” refers to any physical string of monomer units that can be corresponded to a string of nucleotides, including a polymer of nucleotides (e.g., a typical DNA, cDNA or RNA polymer), modified oligonucleotides (e.g., oligonucleotides comprising bases that are not typical to biological RNA or DNA, such as 2′-O-methylated oligonucleotides), and the like. In some embodiments, a nucleic acid can be single-stranded, double-stranded, multi-stranded, or combinations thereof. Unless otherwise indicated, a particular nucleic acid sequence of the presently disclosed subject matter optionally comprises or encodes complementary sequences, in addition to any sequence explicitly indicated.

As used herein, the term “plurality” refers to more than one. Thus, a “plurality of individuals” refers to at least two individuals. In some embodiments, the term plurality refers to more than half of the whole. For example, in some embodiments a “plurality of a population” refers to more than half the members of that population.

As used herein, the term “progeny” refers to the descendant(s) of a particular cross. Typically, progeny result from breeding of two individuals, although some species (particularly some plants and hermaphroditic animals) can be selfed (i.e., the same plant acts as the donor of both male and female gametes). The descendant(s) can be, for example, of the F₁, the F₂, or any subsequent generation.

The term “recipient plant” is used herein to indicate a plant that is to receive DNA obtained from a donor plant that comprises a mutant allele for improved nematode resistance. A “donor plant” is understood within the scope of the invention to mean the plant which provides an alternative or variant allele linked to improved nematode resistance.

As used herein, the phrase “qualitative trait” refers to a phenotypic trait that is controlled by one or a few genes that exhibit major phenotypic effects. Because of this, qualitative traits are typically simply inherited. Examples in plants include, but are not limited to, flower colour, and several known disease resistances such as, for example, Fungus spot resistance or Tomato Mosaic Virus resistance.

“Marker-based selection” is understood within the scope of the invention to refer to e.g. the use of genetic markers to detect one or more nucleic acids from the plant, where the nucleic acid is associated with a desired trait to identify plants that carry genes for desirable (or undesirable) traits, so that those plants can be used (or avoided) in a selective breeding program.

A single nucleotide polymorphism (SNP), a variation at a single site in DNA, is the most frequent type of variation in the genome. A single-nucleotide polymorphism (SNP) is a DNA sequence variation occurring when a single nucleotide—A, T, C, or G—in the genome (or other shared sequence) differs between members of a biological species or paired chromosomes in an individual. For example, two sequenced DNA fragments from different individuals, AAGCCTA to AAGCTTA, contain a difference in a single nucleotide. In this case there are two alleles: C and T. The basic principles of SNP array are the same as the DNA microarray. These are the convergence of DNA hybridization, fluorescence microscopy, and DNA capture. The three components of the SNP arrays are the array that contains nucleic acid sequences (i.e. amplified sequence or target), one or more labelled allele-specific oligonucleotide probes and a detection system that records and interprets the hybridization signal.

The presence or absence of the desired allele may be determined by real-time PCR using double-stranded DNA dyes or the fluorescent reporter probe method.

“PCR (Polymerase chain reaction)” is understood within the scope of the invention to refer to a method of producing relatively large amounts of specific regions of DNA or subset(s) of the genome, thereby making possible various analyses that are based on those regions.

“PCR primer” is understood within the scope of the invention to refer to relatively short fragments of single-stranded DNA used in the PCR amplification of specific regions of DNA.

“Phenotype” is understood within the scope of the invention to refer to a distinguishable characteristic(s) of a genetically controlled trait.

As used herein, the phrase “phenotypic trait” refers to the appearance or other detectable characteristic of an individual, resulting from the interaction of its genome, proteome and/or metabolome with the environment.

“Polymorphism” is understood within the scope of the invention to refer to the presence in a population of two or more different forms of a gene, genetic marker, or inherited trait or a gene product obtainable, for example, through alternative splicing, DNA methylation, etc.

“Selective breeding” is understood within the scope of the invention to refer to a program of breeding that uses plants that possess or display desirable traits as parents.

“Tester” plant is understood within the scope of the invention to refer to a plant used to characterize genetically a trait in a plant to be tested. Typically, the plant to be tested is crossed with a “tester” plant and the segregation ratio of the trait in the progeny of the cross is scored.

“Probe” as used herein refers to a group of atoms or molecules which is capable of recognising and binding to a specific target molecule or cellular structure and thus allowing detection of the target molecule or structure. Particularly, “probe” refers to a labelled DNA or RNA sequence which can be used to detect the presence of and to quantitate a complementary sequence by molecular hybridization.

The term “hybridize” as used herein refers to conventional hybridization conditions, preferably to hybridization conditions at which 5×SSPE, 1% SDS, 1× Denhardts solution is used as a solution and/or hybridization temperature is between 35° C. and 70° C., preferably 65° C. After hybridization, washing is preferably carried out first with 2×SSC, 1% SDS and subsequently with 0.2×SSC at temperatures between 35° C. and 75° C., particularly between 45° C. and 65° C., but especially at 59° C. (regarding the definition of SSPE, SSC and Denhardts solution see Sambrook et al. loc. cit.). High stringency hybridization conditions as for instance described in Sambrook et al, supra, are particularly preferred. Particularly preferred stringent hybridization conditions are for instance present if hybridization and washing occur at 65° C. as indicated above. Non-stringent hybridization conditions for instance with hybridization and washing carried out at 45° C. are less preferred and at 35° C. even less.

In accordance with the present invention, the term “said position corresponding to position X”, X being any number to be found in the respective context in the present application, does not only include the respective position in the SEQ ID NO referred to afterwards but also includes any sequence corresponding to a SmD1 allele or encoding a SmD1 protein, where, after alignment with the reference SEQ ID NO, the respective position might have a different number but corresponds to that indicated for the reference SEQ ID NO. Alignment of SmD1 allelic or SmD1 protein sequences can be effected by applying various alignment tools in a sensible manner, and for example by applying the tools described below.

“Sequence Identity”. The terms “identical” or “identity” in the context of two or more nucleic acid or protein sequences, refer to two or more sequences or sub-sequences that are the same or have a specified percentage of amino acid residues or nucleotides that are the same, when compared and aligned for maximum correspondence, as measured using one of the following sequence comparison algorithms or by visual inspection. If two sequences which are to be compared with each other differ in length, sequence identity preferably relates to the percentage of the nucleotide residues of the shorter sequence which are identical with the nucleotide residues of the longer sequence. As used herein, the percent identity/homology between two sequences is a function of the number of identical positions shared by the sequences (i.e., % identity=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, as described herein below. For example, sequence identity can be determined conventionally with the use of computer programs such as the Bestfit program (Wisconsin Sequence Analysis Package, Version 8 for Unix, Genetics Computer Group, University Research Park, 575 Science Drive Madison, WI 53711). Bestfit utilizes the local homology algorithm of Smith and Waterman, Advances in Applied Mathematics 2 (1981), 482-489, in order to find the segment having the highest sequence identity between two sequences. When using Bestfit or another sequence alignment program to determine whether a particular sequence has for instance 95% identity with a reference sequence of the present invention, the parameters are preferably so adjusted that the percentage of identity is calculated over the entire length of the reference sequence and that homology gaps of up to 5% of the total number of the nucleotides in the reference sequence are permitted. When using Bestfit, the so-called optional parameters are preferably left at their preset (“default”) values. The deviations appearing in the comparison between a given sequence and the above-described sequences of the invention may be caused for instance by addition, deletion, substitution, insertion or recombination. Such a sequence comparison can preferably also be carried out with the program “fasta20u66” (version 2.0u66, September 1998 by William R. Pearson and the University of Virginia; see also W.R. Pearson (1990), Methods in Enzymology 183, 63-98, appended examples and http://workbench.sdsc.edu/). For this purpose, the “default” parameter settings may be used.

Another indication that two nucleic acid sequences are substantially identical is that the two molecules hybridize to each other under stringent conditions. The phrase: “hybridizing specifically to” refers to the binding, duplexing, or hybridizing of a molecule only to a particular nucleotide sequence under stringent conditions when that sequence is present in a complex mixture (e.g., total cellular) DNA or RNA. “Bind(s) substantially” refers to complementary hybridization between a probe nucleic acid and a target nucleic acid and embraces minor mismatches that can be accommodated by reducing the stringency of the hybridization media to achieve the desired detection of the target nucleic acid sequence.

“Stringent hybridization conditions” and “stringent hybridization wash conditions” in the context of nucleic acid hybridization experiments such as Southern and Northern hybridizations are sequence dependent and are different under different environmental parameters. Longer sequences hybridize specifically at higher temperatures. An extensive guide to the hybridization of nucleic acids is found in Tijssen (1993) Laboratory Techniques in Biochemistry and Molecular Biology-Hybridization with Nucleic Acid Probes part I chapter 2 “Overview of principles of hybridization and the strategy of nucleic acid probe assays” Elsevier, New York. Generally, highly stringent hybridization and wash conditions are selected to be about 5° C. lower than the thermal melting point for the specific sequence at a defined ionic strength and pH. Typically, under “stringent conditions” a probe will hybridize to its target subsequence, but to no other sequences.

The “thermal melting point” is the temperature (under defined ionic strength and pH) at which 50% of the target sequence hybridizes to a perfectly matched probe. Very stringent conditions are selected to be equal to the melting temperature (T.sub.m) for a particular probe. An example of stringent hybridization conditions for hybridization of complementary nucleic acids which have more than 100 complementary residues on a filter in a Southern or northern blot is 50% formamide with 1 mg of heparin at 42° C., with the hybridization being carried out overnight. An example of highly stringent wash conditions is 0.15M NaCl at 72° C. for about 15 minutes. An example of stringent wash conditions is a 0.2 times SSC wash at 65° C. for 15 minutes (see, Sambrook, infra, for a description of SSC buffer). Often, a high stringency wash is preceded by a low stringency wash to remove background probe signal. An example medium stringency wash for a duplex of, e.g., more than 100 nucleotides, is 1 times SSC at 45° C. for 15 minutes. An example low stringency wash for a duplex of, e.g., more than 100 nucleotides, is 4-6 times SSC at 40° C. for 15 minutes. For short probes (e.g., about 10 to 50 nucleotides), stringent conditions typically involve salt concentrations of less than about 1.0M Na ion, typically about 0.01 to 1.0 M Na ion concentration (or other salts) at pH 7.0 to 8.3, and the temperature is typically at least about 30° C. Stringent conditions can also be achieved with the addition of destabilizing agents such as formamide. In general, a signal to noise ratio of 2 times (or higher) than that observed for an unrelated probe in the particular hybridization assay indicates detection of a specific hybridization. Nucleic acids that do not hybridize to each other under stringent conditions are still substantially identical if the proteins that they encode are substantially identical. This occurs, e.g. when a copy of a nucleic acid is created using the maximum codon degeneracy permitted by the genetic code.

### Plants, Seeds, Fruits

In a first embodiment, the invention provides a plant comprising a SmD1 allele encoding a SmD1 protein having at least 90% or 91%, preferably 92%, 93% or 94%, more preferably 95%, 96% or 97%, even more preferably 98% or 99% amino acid sequence identity with SEQ ID NO: 1, wherein said SmD1 protein comprises a missense mutation resulting in a modified SmD1 protein conferring an improved nematode resistance.

In a further embodiment, said modified SmD1 protein comprises a missense mutation at a position corresponding to any one of amino acid positions 1 to 108 of SEQ ID NO: 1.

In a further embodiment, said modified SmD1 protein comprises a missense mutation at a position corresponding to amino acid position 14 of SEQ ID NO: 1.

In a further embodiment, said modified SmD1 protein comprises a threonine to isoleucine substitution at a position corresponding to amino acid position 14 of SEQ ID NO: 1.

In a further embodiment, the invention provides a plant according to any of the preceding embodiments, wherein said SmD1 allele is a SmD1b allele.

In a further embodiment, the invention provides a plant according to the preceding embodiments wherein said SmD1 allele encoding said modified SmD1 protein is artificially created. In a further embodiment, the invention provides a plant according to the preceding embodiments wherein said plant is not exclusively obtained by an essentially biological process.

In a further embodiment, the invention provides a plant according to any of the preceding embodiments, wherein said SmD1 allele has at least 60%, 65%, 70%, 75%, 80%, 85%, 90% or 95% nucleic acid sequence identity with SEQ ID NO: 20.

In a further embodiment, the invention provides a plant according to any of the preceding embodiments, wherein said plant is selected from the list comprising tomato, tobacco, pepper, squash, watermelon, melon, cucumber and soy.

In a further embodiment, the invention provides a plant according to any of the preceding embodiments, wherein said plant is an inbred, a dihaploid or a hybrid plant.

In another embodiment, the plant according to the invention is male sterile. In another embodiment, the plant according to the invention is cytoplasmic male sterile.

In a further embodiment, the invention provides a plant according to any of the preceding embodiments, wherein said plant is a rootstock.

In a further embodiment, the invention provides a plant according to any of the preceding embodiments, wherein said plant comprises two copies of said SmD1 allele.

In a further embodiment, said modified SmD1 protein confers an intermediate resistance against the nematodes of the genera Meloidogyne, Heterodera and Globodera, preferably Meloidogyne, more preferably Meloidogyne incognita, Meloidogyne arenaria, Meloidogyne hapla, Meloidogyne enterolobii and Meloidogyne javanica.

In a further embodiment, the invention provides a plant according to any of the preceding embodiments, wherein said plant is Solanum lycopersicum.

In a further embodiment, the invention provides a plant according to any of the preceding embodiments, wherein said modified SmD1 protein has an amino acid sequence of SEQ ID NO: 2.

In a further embodiment, the invention provides a plant according to any of the preceding embodiments, wherein said SmD1b allele comprises the nucleic acid sequence of SEQ ID NO: 19 or SEQ ID NO: 21. In a further embodiment, the invention provides a plant according to any of the preceding embodiments, wherein said SmD1b allele consists of the nucleic acid sequence of SEQ ID NO: 19 or SEQ ID NO: 21.

In a further embodiment, the invention provides a plant according to any of the preceding embodiments, wherein said SmD1b allele is obtainable from Solanum lycopersicum line 19TEP250122, deposited with NCIMB on 29 Nov. 2019 under NCIMB Accession No. 43529.

In a further embodiment, the invention provides a plant according to any of the preceding embodiments, wherein in case of nematode infestation, the number of females with egg masses is decreased by 25%, preferably by 50%, when compared with the same cultivated plant lacking said SmD1 allele.

It is a further embodiment to provide a plant part, organ or tissue obtainable from a cultivated plant, preferably a cultivated tomato plant, more preferably a cultivated Solanum lycopersicum plant according to any of preceding embodiments, including but not limiting to leaves, stems, roots, flowers or flower parts, fruits, shoots, gametophytes, sporophytes, pollen, anthers, microspores, egg cells, zygotes, embryos, meristematic regions, callus tissue, seeds, cuttings, cell or tissue cultures or any other part or product of the plant which still exhibits the improved nematode resistance trait according to the invention, particularly when grown into a plant that produces fruits.

In a further embodiment, the invention provides fruit produced by a plant according to any of the preceding embodiments. In a further embodiment, the invention provides tomato fruit produced by a tomato plant according to any of the preceding embodiments.

In a further embodiment, the invention provides a seed that produces the plant of any of the preceding embodiments. In a further embodiment, the invention provides a tomato seed that produces a tomato plant according to any of the preceding embodiments.

### Alleles, Markers

The present invention is further directed to a mutant SmD1 allele, preferably a mutant SmD1b allele associated with the nematode resistance trait in the plant. In a further embodiment, the present invention is directed to a mutant SmD1 allele, of which the wild type version is SEQ ID NO: 20, encoding the SmD1 protein of SEQ ID NO: 1, or of which a wild type SmD1 allele encoding a SmD1 protein having at least 90% amino acid sequence identity to SEQ ID NO: 1, wherein said mutant SmD1 allele encodes a modified SmD1 protein having a missense mutation resulting in an improved nematode resistance phenotype. In a further embodiment, SEQ ID NO: 21 is a mutant SmD1 allele encoding the modified SmD1 protein of SEQ ID NO: 2. In a further embodiment, the tomato SmD1 allele of the present invention is located on chromosome 9. In a further embodiment of the present invention, one tomato SmD1b allele according to the invention is obtainable, obtained or derived from a donor plant which is Solanum lycopersicum line 19TEP250122, deposited with NCIMB on 29 Nov. 2019 under NCIMB Accession No. 43529, or a progeny or an ancestor thereof, and comprising said one SmD1b allele of the invention.

In a further embodiment, the invention relates to an isolated nucleic acid sequence encoding SEQ ID NOs: 1 or 2. In a further embodiment, said isolated nucleic acid sequence is SEQ ID NO: 18, 19, 20 or 21.

The present invention discloses a kit for the detection of the nematode resistance trait allele in a cultivated tomato plant, particularly a cultivated Solanum lycopersicum plant, wherein said kit comprises one PCR oligonucleotide primer pair represented by a forward primer of SEQ ID NO: 22 and a reverse primer of SEQ ID NO: 23. This kit allows for the detection of a SmD1b allele of the invention, wherein the resulting amplicon is sequenced and the T14I (ACT-->ATT codon) mutation of the present invention is detected. In this context, the T14I mutation can be used as a SNP marker.

The present invention also discloses the use of SNP markers according to the invention for diagnostic selection and/or genotyping of the nematode resistance trait allele in a cultivated plant, particularly a cultivated tomato plant, more particularly a cultivated Solanum lycopersicum plant.

The present invention further discloses the use of SNP markers according to the invention for identifying in a plant, particularly a cultivated tomato plant, more particularly a Solanum lycopersicum plant according to the invention, the presence of the nematode resistance trait allele and/or for monitoring the introgression of the nematode resistance trait allele in a cultivated plant, particularly a cultivated tomato plant, more particularly a Solanum lycopersicum plant according to the invention and as described herein.

The invention further discloses a polynucleotide (amplification product) obtainable in a PCR reaction involving one oligonucleotide primer or a pair of PCR oligonucleotide primers of SEQ ID NO 22 and SEQ ID NO 23 that is statistically correlated and thus co-segregates with the nematode resistance trait or with one of the markers disclosed, which amplification product corresponds to an amplification product obtainable from Solanum lycopersicum line 19TEP250122, deposited with NCIMB on 29 Nov. 2019 under NCIMB Accession No. 43529, or a progeny or an ancestor thereof, comprising the SmD1b allele of the invention, in a PCR reaction with identical primers or primer pairs provided that the respective allele is still present in said plant and/or can be considered an allele thereof.

Also contemplated herein is a polynucleotide that has at least 60%, particularly at least 65%, particularly at least 70%, particularly at least 75%, particularly at least 80%, particularly at least 85%, particularly at least 90%, particularly at least 95%, sequence identity with the sequence of said amplification product and/or a polynucleotide exhibiting a nucleotide sequence that hybridizes to the nucleotide sequences of said amplification product obtainable in the above PCR reaction.

The amplification product according to the invention and described herein above can then be used for generating or developing new primers and/or probes that can be used for identifying the nematode resistance trait allele.

The present invention therefore further relates in one embodiment to derived markers, particularly to derived primers or probes, developed from an amplification product according to the invention and as described herein above by methods known in the art, which derived markers are genetically linked to the improved nematode resistance trait locus.

The present invention also relates to a method of identifying a cultivated tomato plant, preferably a cultivated Solanum lycopersicum plant, exhibiting improved nematode tolerance and having at least one copy of a SmD1 allele encoding a SmD1 protein having at least 90% amino acid sequence identity with SEQ ID NO: 1, wherein said SmD1 protein comprises a missense mutation resulting in a modified SmD1 protein, comprising the steps of:
a) Obtaining a population of mutant plants;
b) Screening said population for the presence of said SmD1 allele.

### Methods of Breeding

In another embodiment the invention relates to a method of providing a cultivated plant, preferably a cultivated tomato plant, more preferably a cultivated plant Solanum lycopersicum, plant part or seed, wherein said method comprises the following steps:
a) Crossing a 1^{st} plant according to any of the preceding embodiments with a 2^{nd} plant lacking the SmD1 allele of the invention,
b) Obtaining a progeny plant, and,
c) Optionally, selecting a plant of said progeny characterized in that said plant exhibits an improved nematode resistance.

In a further embodiment, the invention provides a method for producing a cultivated plant, preferably a cultivated tomato plant, more preferably a cultivated Solanum lycopersicum plant, exhibiting improved nematode resistance comprising the steps of
a) crossing a 1^{st} plant according to any of the preceding embodiments comprising at least one copy of an SmD1 allele of the invention with a 2^{nd} cultivated plant lacking said SmD1 allele;
b) Selecting a progeny plant exhibiting an improved nematode resistance;
   wherein the selection of step b) is carried out by detecting the presence of an SmD1 allele of the invention with the primer pair of SEQ ID NOs 22 and 23, following by sequencing of the resulting amplicon.

In a further embodiment the invention relates to the method of any of the preceding embodiments wherein the 1^{st} plant of step a) is Solanum lycopersicum line 19TEP250122, deposited with NCIMB on 29 Nov. 2019 under NCIMB Accession No. 43529.

In another embodiment the invention relates to a method of providing a cultivated plant, preferably a cultivated tomato plant, more preferably a cultivated Solanum lycopersicum plant, exhibiting improved nematode resistance comprising the steps of:
a) Crossing a 1^{st} plant according to any of the preceding embodiments with a 2^{nd} plant lacking an SmD1 allele of the invention,
b) Obtaining a progeny cultivated plant, and,
c) Optionally, selecting a plant of said progeny characterized in that said plant, in case of nematode infestation, exhibits a number of female with egg masses which is decreased by 25%, preferably by 50%, when compared with the same cultivated plant lacking said SmD1 allele.

In a further embodiment is considered the method of any of the preceding embodiments wherein the 1^{st} tomato plant of step a) is Solanum lycopersicum line 19TEP250122, deposited with NCIMB on 29 Nov. 2019 under NCIMB Accession No. 43529, or a progeny or an ancestor thereof.

In another embodiment is considered a method for producing a cultivated plant, preferably a cultivated tomato plant, more preferably a cultivated Solanum lycopersicum plant, exhibiting improved nematode resistance comprising the following steps:
a) Providing seeds of a plant according to any of the previous embodiments,
b) Germinating said seed and growing a mature, fertile plant therefrom,
c) Inducing self-pollination of said plant under a), growing fruits and harvesting the fertile seeds therefrom, and
d) Growing plants from the seeds harvested under c) and selecting an improved nematode resistance plant.

It is a further embodiment of the present invention to provide a method for providing plants exhibiting an improved nematode resistance by introducing into a plant a nucleotide sequence encoding a SmD1 protein having at least 90% amino acid sequence identity with SEQ ID NO: 1, wherein said SmD1 protein comprises a missense mutation resulting in a modified SmD1 protein conferring an improved nematode resistance. It is a further embodiment of the present invention to provide a method for providing tomato plants exhibiting an improved nematode resistance by introducing into a tomato plant a nucleotide sequence encoding a SmD1 protein of SEQ ID NO: 2. It is a further embodiment of the present invention to provide a method for providing tomato plants exhibiting an improved nematode resistance by introducing into a tomato plant a nucleotide sequence of SEQ ID NO: 19 or 21.

In a further embodiment, the invention provides a method of improving nematode resistance in a plant, comprising the steps of
a) Obtained a population of mutant plants;
b) Selecting a mutant plant comprising a modified SmD1b allele encoding a SmD1 protein with a missense mutation in its amino acid sequence.

The modified SmD1 alleles can also be introduced by way of mutagenesis, for example by way a chemical mutagenesis, for example by way of EMS mutagenesis. Alternatively, or subsequently, the modified SmD1 alleles can be identified and/or introduced by way of using tilling techniques.

The modified SmD1 alleles can also be introduced by targeted mutagenesis, e.g. by way of homologous recombination, zinc-finger nucleases, oligonucleotide-based mutation induction, transcription activator-like effector nucleases (TALENs), clustered regularly interspaced short palindromic repeat (CRISPR) systems or any alternative technique to edit the genome.

Alternatively, the modified SmD1b alleles can also be introduced by transgenic or cis-genic methods via a nucleotide construct which may be comprised in a vector.

### Uses

In another embodiment the invention relates to the use of a cultivated plant, preferably a cultivated tomato plant, more preferably a cultivated Solanum lycopersicum plant, plant part or seed according to any of the preceding embodiments for growing a plant and producing and harvesting crops and/or fruits.

In another embodiment the invention relates to the use of a cultivated plant, preferably a cultivated tomato plant, more preferably a cultivated Solanum lycopersicum plant, according to any of the preceding embodiments for producing fruits for the fresh market or for food processing.

In another embodiment the invention relates to the use of a cultivated tomato plant, preferably a cultivated Solanum lycopersicum plant, plant part or seed according to any of preceding embodiments, wherein the cultivated tomato plant, preferably the cultivated Solanum lycopersicum plant, plant part or seed is Solanum lycopersicum line 19TEP250122, deposited with NCIMB on 29 Nov. 2019 under NCIMB Accession No. 43529, or a progeny or an ancestor thereof.

In a further embodiment the invention relates to the use of a cultivated plant, preferably a cultivated tomato plant, more preferably a cultivated Solanum lycopersicum plant, plant part or seed according to any of the preceding embodiments to sow a field, a greenhouse, or a plastic house. In a further embodiment the invention relates to the use of a cultivated plant, preferably a cultivated tomato plant, more preferably a cultivated Solanum lycopersicum plant, plant part or seed according to any of the preceding embodiments as a rootstock plant.

The present invention also relates to the use of nematode resistant-propagating material obtainable from a plant according to any of the preceding embodiments for growing a plant, wherein said nematode resistance may be assessed in a standard assay, particularly an assay as described in Example 4 below.

In a further embodiment the invention relates to the use of a SmD1 allele of the invention to confer the improved nematode resistance trait to a plant lacking said allele.

The invention further relates to the use of a plant according to any of the preceding embodiments to introgress a nematode resistance trait into a plant lacking said trait.

Based on the description of the present invention, the skilled person who is in possession of Solanum lycopersicum line 19TEP250122, deposited with NCIMB on 29 Nov. 2019 under NCIMB Accession No. 43529, or a progeny or an ancestor thereof, comprising one copy of a SmD1 allele according to the invention, as described herein, has no difficulty to transfer said allele of the present invention to other tomato plants of various types using breeding techniques well-known in the art. Alternatively, based on the description of the present invention, including the disclosure of SmD1 alleles having a missense mutation resulting in modified SmD1 protein associated with a nematode tolerance phenotype, the skilled person has no difficulty to reproduce the present invention, using techniques well-known in the art.

### Seed Deposit Details

Applicant has made a deposit of 2500 seeds of Solanum lycopersicum line 19TEP250122 with NCIMB on 29 Nov. 2019 under NCIMB Accession No. 43529. The deposited seeds were obtained from a population segregating for the SmD1 allele of the invention. Consequently, 50% of the deposited seeds are homozygous for the mutant allele, 25% of the deposited seeds are heterozygous for the mutant allele and 25% of the seeds are homozygous for the wild type SmD1 allele.

Applicant requests that the deposited material be released only to an Expert according to Rule 32(1) EPC or corresponding laws and rules of other countries or treaties (Expert Witness clause), until the mention of the grant of the patent publishes, or from 20 years from the date of filing if the application is refused, withdrawn or deemed to be withdrawn.

### EXAMPLES

### Example 1: Identification of Plant Protein SmD1 as a Target for Nematode Effectors

It was observed in a yeast two-hybrid experiment using M. incognita effector MiEFF18 (Minc18636; Nguyen et al. 2018) as a bait that the tomato plant protein SmD1 was a potential target of the nematode effector. This interaction was first demonstrated in tomato where the SmD1 protein is 100% identical (SEQ ID NOs: 1 and 3) for the two tomato SmD1 genes Sl06g084310.2.1 and Sl09g064660.2.1. The interaction was then validated in Arabidopsis thaliana for the SmD1b protein (SEQ ID NO: 4) from Arabidopsis gene AT4G02840. The captured SmD1 protein and their corresponding genes are shown in Table 1.

TABLE 1
Identifiers of the tomato and Arabidopsis respective orthologous
genes encoding the conserved SmD1 and SmD1b protein, respectively.

| | Captured | Tomato | Captured | Arabidopsis |
|---|---|---|---|---|
| M. incognita | protein in | SmD1 | protein in | SmD1b |
| effector | tomato | genes | Arabidopsis | gene |
| Minc18636 = | Spliceosomal | SI06g084310.2.1 | Spliceosomal | AT4G02840 |
| Mi-EFF18 | SmD1 | SI09g064660.2.1 | SmD1b | |

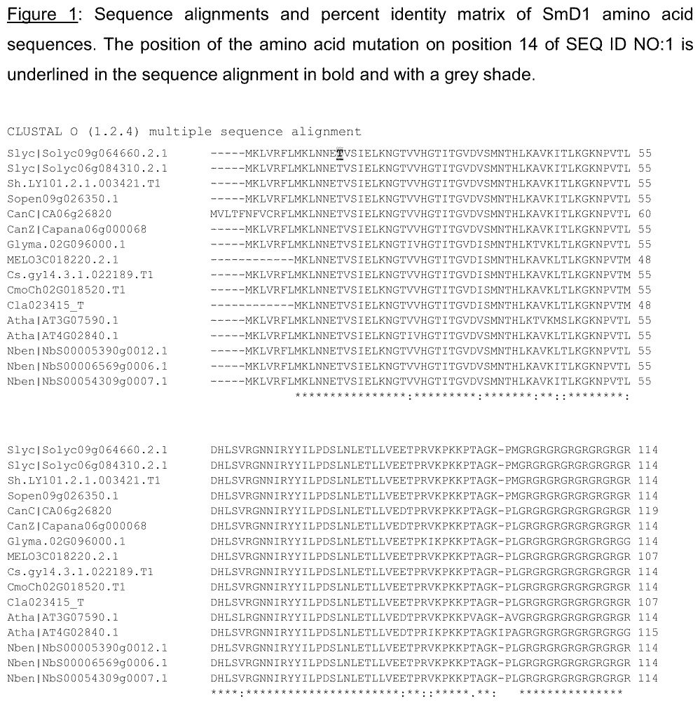

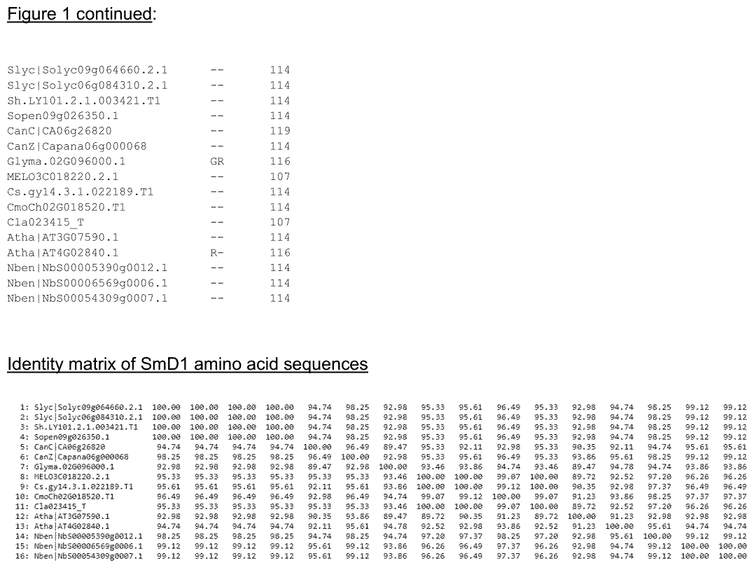

In the yeast two-hybrid experiment, the portion of the SmD1 protein interacting with the nematode effector was shown to be the first 108 amino acids. FIG. 1 discloses an alignment of the SmD1 amino acid sequences highlighting the high degree of conservation amongst plant species.

### Example 2A: Effect of Arabidopsis Smd1 Mutations on Nematode Susceptibility

In order to validate the role of the SmD1 gene in the susceptibility to nematode, Arabidopsis smd1a (AT3G07590) and smd1b (AT4G02840) mutant plants (in Columbia background) were retrieved (Elvira-Matelot et al., 2016) and assessed for their level of susceptibility when subjected to M. incognita.

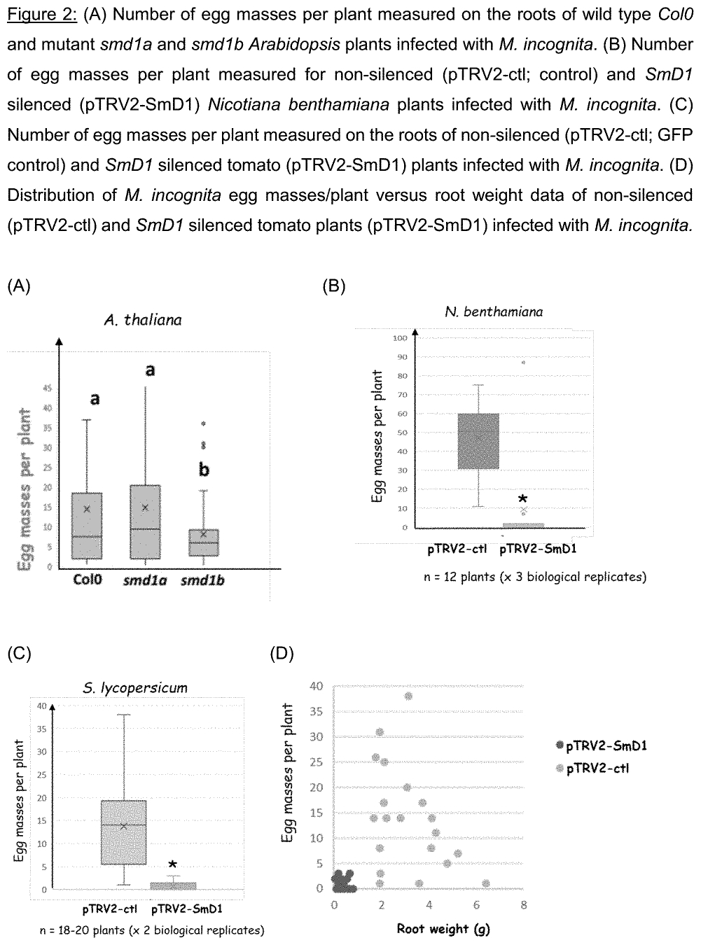

It was found that Arabidopsis smd1b mutants are significantly less susceptible to M. incognita when compared with the wild type Columbia plant or the smd1a mutant (FIG. 2A), suggesting that AtSmD1b is primarily involved in the nematode susceptibility mechanisms.

### Example 2B: Effect of Nicotiana benthamiana SmD1 Silencing on Nematode Susceptibility

In order to confirm the important role of the SmD1 gene in the susceptibility to nematode, Nicotiana benthamiana plants silenced for their SmD1 genes were generated and assessed for their level of susceptibility when subjected to M. incognita.

It was again found that Nicotiana benthamiana plants which are silenced for their SmD1 genes were significantly less susceptible to M. incognita when compared with the control tobacco plant (FIG. 2B), suggesting that NbSmD1 genes are similarly involved in the nematode susceptibility mechanisms.

### Example 2C: Effect of Tomato SmD1 Silencing on Nematode Susceptibility

Finally, the important role of the SmD1 gene in the susceptibility to nematode was also confirmed in tomato. Tomato plants silenced for their SmD1 genes were generated in the Saint-Pierre background and assessed for their level of susceptibility when subjected to M. incognita. It was again found that tomato plants silenced for their SmD1 genes were significantly less susceptible to M. incognita when compared with the control tomato plant (FIG. 2C), suggesting that SlSmD1 genes are similarly involved in the nematode susceptibility mechanisms.

However, the tomato plants silenced for their SlSmD1 genes also presented a commercially adverse phenotype with a significantly reduced root system (FIG. 2D), general dwarfism and ultimately a lower fruit yield. Consequently, plants modified with a nonsense mutation or a KO-type mutation in their SmD1 genes, albeit being more resistant to nematode, are likely to exhibit unwanted characteristics due to the absence of a working version of the SmD1 protein in planta.

### Example 3: Identification of a Commercially Relevant Tomato SmD1b Mutant

To obtain a tomato plant exhibiting an increased resistance to nematode while preserving the economic value of the crop, mutants were generated in the M82 background using EMS and screened in a tilling approach to identify tomato plants with a modified SmD1 gene resulting in a missense mutation of the SmD1 protein. One resulting tilling tomato line having a missense mutation in its SmD1b gene (line #123, 18TEP250123, homozygous for the mutation, ancestor plant of deposited line 19TEP250122, with a missense mutation at position 14 of SEQ ID NO:1) was inoculated with M. incognita and female forming egg masses and root weight were determined six weeks after infection, and compared with a control M82 line #117 (18TEP250117, +/+, WT).

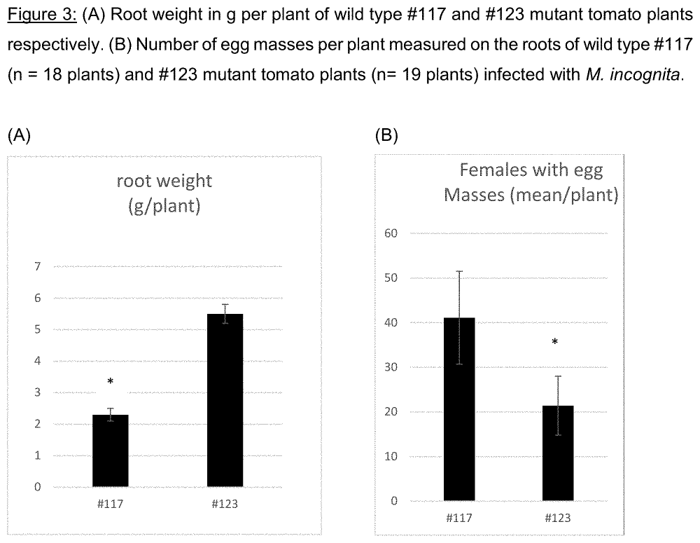

The analysis of the morphology and the weight of the roots show a net increase of the root system of the #123 mutant line (FIG. 3 (A)). In the same time, the #123 mutant line showed a significant decrease (Mann-Whitney test, α=2.5%) of 50% in the number of females forming egg masses (FIG. 3 (B)).

The genotyping of 6 plants of each line was performed in order to verify the homozygosity of the mutation in SmD1b (Solyc09g064660) using primer SISmD1b-M82-F (ATTTTGAACAACCCCTGGCG (SEQ ID NO: 22)) and SISmD1b-M82-R (ACTCTACGACCTCACCACTT (SEQ ID NO: 23)). The sequencing results of the 420 bp amplicons showed that all #117 plants have a wild-type SmD1b allele while all #123 plants have a homozygous SmD1b mutant allele (ACT-->ATT codon) that leads to a missense mutation (T14I).

TABLE 2
Position of the mutation in the genomic, coding and protein sequences.

| Mutation | Mutation | | Mutation | |
|---|---|---|---|---|
| position on | position on | | position on | |
| genomic DNA | coding sequence | Mutation | protein sequence | Mutation |
| SEQ ID NOs: 20/21 | SEQ ID NOs: 18/19 | (gene) | SEQ ID NOs: 1/2 | (protein) |
| 296 | 41 | ACT −> ATT | 14 | T14I |
| | | | | (nonsense |
| | | | | mutation) |

In conclusion, it was shown that SmD1b missense mutation (T14I) leads to an increased resistance to the root-knot nematode M. incognita while preserving the function of the SmD1 protein in planta. Given the much-conserved structure of the SmD1 protein amongst plant species, it is expected that similar missense mutations in orthologous SmD1b genes will provide similar effects to those observed with the #123 mutant tomato line.

### Example 4: Protocol to Assess Nematode Tolerance in Tomato Plants

Meloidogyne incognita (Calissane strain) are multiplied in tomato plants (Solanum lycopersicum cv St Pierre) in greenhouse. Freshly hatched second-stage juveniles (J2s) were collected as described previously (Caillaud and Favery, 2016). Sterile tomato seeds (cv M82) are sown in soil mixed with sand (1:1); after 48 hours at 4° C., samples were transferred in a growth chamber with 16 h photoperiod at 24° C. 7-days old plantlets were transferred to small pots in soil/sand individually. One-month old tomato seedlings were inoculated with 150 M. incognita J2s per plant. Roots were collected 6 weeks after infection and stained with eosin 0.5%. Females forming egg masses and root weight were determined 6 weeks after infection.

### BIBLIOGRAPHY

Caillaud and Favery, 2016, In vivo imaging of microtubule organization in dividing giant cell. In Plant Cell Division: Methods and Protocols, Methods in Molecular Biology, Marie-Cécile Caillaud (ed.), Springer Science+Business Media New York, vol. 1370, DOI 10.1007/978-1-4939-3142-2_11.
Elvira-Matelot et al., 2016, The nuclear ribonucleoprotein SmD1 interplays with splicing, RNA quality control, and posttranscriptional gene silencing in Arabidopsis, The Plant Cell 28(2), DOI: 10.1105/tpc.15.01045.
Kiewnick et al., 2009, Effects of the Mi-1 and the N root-knot nematode-resistance gene on infection and reproduction of Meloidogyne enterolobii on tomato and pepper cultivars, J. Nematol. 41(2), pages 134-139.
Mejias et al., 2019, Plant proteins and processes targeted by parasitic nematode effectors, Front. Plant Sci. July 2019 10:970, doi: 10.3389/fpls.2019.00970, eCollection 2019.
Nguyen et al., 2018, A root-knot nematode small glycine and cysteine-rich secreted effector, MiSGCR1, is involved in plant parasitism. New Phytol., 217: 687-699. Doi: 10.1111/nph.14837.
Sievers et al., 2011, Fast, scalable generation of high-quality protein multiple sequence alignments using Clustal Omega, Mol. Syst. Biol. 2011; 7: 539. https://www.ebi.ac.uk/Tools/msa/clustalo/
Singh et al., 2013, Plant-parasitic nematodes of potential phytosanitary importance, their main hosts and reported yield losses, EPPO Bulletin 43(2), pages 334-374.

1. A plant comprising a SmD1 allele encoding a SmD1 protein having at least 90% amino acid sequence identity with SEQ ID NO: 1, wherein said SmD1 protein comprises a missense mutation resulting in a modified SmD1 protein conferring an improved nematode resistance.

2. The plant of claim 1, wherein said modified SmD1 protein comprises a missense mutation at a position corresponding to any one of amino acid positions 1 to 108 of SEQ ID NO: 1.

3. The plant of claim 1, wherein said modified SmD1 protein comprises a missense mutation at a position corresponding to amino acid position 14 of SEQ ID NO: 1.

4. The plant of claim 1, wherein said modified SmD1 protein comprises a threonine to isoleucine substitution at a position corresponding to amino acid position 14 of SEQ ID NO: 1.

5. The plant of claim 1, wherein said SmD1 allele is obtained by way of mutagenesis.

6. The plant of claim 1, wherein said plant is selected from the list comprising tomato, tobacco, pepper, squash, watermelon, melon, cucumber and soy.

7. The plant of claim 6, wherein said plant is an inbred, a dihaploid or a hybrid plant.

8. The plant of claim 6, wherein said plant is a rootstock.

9. The plant of claim 1, wherein said plant comprises two copies of said SmD1 allele.

10. The plant of claim 1, wherein said modified SmD1 protein confers an improved resistance against the nematodes of the genera Meloidogyne, preferably Meloidogyne incognita, Meloidogyne arenaria, Meloidogyne hapla, Meloidogyne enterolobii and Meloidogyne javanica.

11. The plant of claim 1, wherein said plant is Solanum lycopersicum.

12. The plant of claim 11 wherein said modified SmD1 protein has an amino acid sequence of SEQ ID NO: 2.

13. The Solanum lycopersicum plant of claim 12, wherein said SmD1 allele is obtainable from Solanum lycopersicum line 19TEP250122, deposited with NCIMB on 29 Nov. 2019 under NCIMB Accession No. 43529.

14. A plant part of the plant of claim 1, wherein said plant part comprises said SmD1 allele.

15. A seed that produces the plant of claim 1.

16. A method of improving nematode resistance in plant, comprising the steps of
a) Obtaining a population of mutant plants;
b) Selecting a mutant plant comprising a modified SmD1 allele encoding a SmD1 protein with a missense mutation in its amino acid sequence.

17. A method of identifying a cultivated tomato plant, preferably a cultivated Solanum lycopersicum plant, exhibiting improved nematode tolerance and having at least one copy of a SmD1 allele encoding a SmD1 protein having at least 90% amino acid sequence identity with SEQ ID NO: 1, wherein said SmD1 protein comprises a missense mutation resulting in a modified SmD1 protein, comprising the steps of:
a) Obtaining a population of mutant plants;
b) Screening said population for the presence of said SmD1 allele.

18. A kit for the detection of a nematode resistance trait SmD1 allele in a cultivated tomato plant, particularly a cultivated Solanum lycopersicum plant, wherein said kit comprises one PCR oligonucleotide primer pair represented by a forward primer of SEQ ID NO: 22 and a reverse primer of SEQ ID NO: 23.


SEQUENCE LISTING (35 sequences)

SEQ ID NO: 1 - protein, 114 aa, Solanum lycopersicum
MKLVRFLMKLNNETVSIELKNGTVVHGTITGVDVSMNTHLKAVKITLKGKNPVTLDHLSVRGNNIRYYILPDSLNLETLLVEETPRVKPKKPTAGKPMGRGRGRGRGRGRGRGR

SEQ ID NO: 2 - protein, 114 aa, Solanum lycopersicum
MKLVRFLMKLNNEIVSIELKNGTVVHGTITGVDVSMNTHLKAVKITLKGKNPVTLDHLSVRGNNIRYYILPDSLNLETLLVEETPRVKPKKPTAGKPMGRGRGRGRGRGRGRGR

SEQ ID NO: 3 - protein, 114 aa, Solanum lycopersicum; identical to SEQ ID NO: 1

SEQ ID NO: 4 - protein, 116 aa, Arabidopsis thaliana
MKLVRFLMKLNNETVSIELKNGTIVHGTITGVDVSMNTHLKAVKLTLKGKNPVTLDHLSVRGNNIRYYILPDSLNLETLLVEDTPRIKPKKPTAGKIPAGRGRGRGRGRGRGRGGR

SEQ ID NO: 5 - protein, 114 aa, Arabidopsis thaliana
MKLVRFLMKLNNETVSIELKNGTVVHGTITGVDVSMNTHLKTVKMSLKGKNPVTLDHLSLRGNNIRYYILPDSLNLETLLVEDTPRVKPKKPVAGKAVGRGRGRGRGRGRGRGR

SEQ ID NO: 6 - protein, 114 aa, Nicotiana benthamiana
MKLVRFLMKLNNETVSIELKNGTVVHGTITGVDVSMNTHLKAVKLTLKGKNPVTLDHLSVRGNNIRYYILPDSLNLETLLVEETPRVKPKKPTAGKPLGRGRGRGRGRGRGRGR

SEQ ID NO: 7 - protein, 114 aa, Nicotiana benthamiana
MKLVRFLMKLNNETVSIELKNGTVVHGTITGVDVSMNTHLKAVKITLKGKNPVTLDHLSVRGNNIRYYILPDSLNLETLLVEETPRVKPKKPTAGKPLGRGRGRGRGRGRGRGR

SEQ ID NO: 8 - protein, 114 aa, Nicotiana benthamiana; identical to SEQ ID NO: 7

SEQ ID NO: 9 - protein, 116 aa, Glycine max
MKLVRFLMKLNNETVSIELKNGTIVHGTITGVDISMNTHLKTVKLTLKGKNPVTLDHLSVRGNNIRYYILPDSLNLETLLVEETPKIKPKKPTAGKPLGRGRGRGRGRGRGRGGGR

SEQ ID NO: 10 - protein, 119 aa, Capsicum annuum
MVLTFNFVCRFLMKLNNETVSIELKNGTVVHGTITGVDVSMNTHLKAVKITLKGKNPVTLDHLSVRGNNIRYYILPDSLNLETLLVEDTPRVKPKKPTAGKPLGRGRGRGRGRGRGRGR

SEQ ID NO: 11 - protein, 114 aa, Capsicum annuum
MKLVRFLMKLNNETVSIELKNGTVVHGTITGVDVSMNTHLKAVKITLKGKNPVTLDHLSVRGNNIRYYILPDSLNLETLLVEDTPRVKPKKPTAGKPLGRGRGRGRGRGRGRGR

SEQ ID NO: 12 - protein, 114 aa, Cucurbita moschata
MKLVRFLMKLNNETVSIELKNGTVVHGTITGVDISMNTHLKAVKLTLKGKNPVTMDHLSVRGNNIRYYILPDSLNLETLLVEETPRVKPKKPTAGKPLGRGRGRGRGRGRGRGR

SEQ ID NO: 13 - protein, 107 aa, Cucumis melo
MKLNNETVSIELKNGTVVHGTITGVDISMNTHLKAVKLTLKGKNPVTMDHLSVRGNNIRYYILPDSLNLETLLVEETPRVKPKKPTAGRPLGRGRGRGRGRGRGRGR

SEQ ID NO: 14 - protein, 114 aa, Cucumis sativus
MKLVRFLMKLNNETVSIELKNGTVVHGTITGVDISMNTHLKAVKLTLKGKNPVTMDHLSVRGNNIRYYILPDSLNLETLLVEETPRVKPKKPTAGRPLGRGRGRGRGRGRGRGR

SEQ ID NO: 15 - protein, 107 aa, Citrullus lanatus; identical to SEQ ID NO: 13

SEQ ID NO: 16 - protein, 114 aa, Solanum habrochaites; identical to SEQ ID NO: 1

SEQ ID NO: 17 - protein, 114 aa, Solanum pennellii; identical to SEQ ID NO: 1

SEQ ID NO: 18 - DNA, 345 bp, Solanum lycopersicum
atgaagctcgtcagatttttgatgaagctgaacaacgagactgtctcaattgagctcaaaaacggcaccgttgttcatggaaccattacaggtgtggatgttagcatgaacacacatctgaaggctgtcaaaattacgctaaaaggaaagaatccagtgacattggatcacctgagtgtgaggggtaacaacatccgttattacatcctccctgacagcttaaatcttgagacgttactggtggaagaaacacctagggtgaagccaaagaagccaacagctggaaagcctatgggacgtggtcgtgggcgcggtcgtgggcgtggacgtggtcgaggccgctaa

SEQ ID NO: 19 - DNA, 345 bp, Solanum lycopersicum
atgaagctcgtcagatttttgatgaagctgaacaacgagattgtctcaattgagctcaaaaacggcaccgttgttcatggaaccattacaggtgtggatgttagcatgaacacacatctgaaggctgtcaaaattacgctaaaaggaaagaatccagtgacattggatcacctgagtgtgaggggtaacaacatccgttattacatcctccctgacagcttaaatcttgagacgttactggtggaagaaacacctagggtgaagccaaagaagccaacagctggaaagcctatgggacgtggtcgtgggcgcggtcgtgggcgtggacgtggtcgaggccgctaa

SEQ ID NO: 20 - DNA, 4172 bp, Solanum lycopersicum
tcttatattcttagcattaaatagtgtgagacgacggcccacgtttagtccaatggacgttcccagacgaggcatataataagacatcacttgtgtattttgaacaacccctggcgctccttgccctaaactaaagccctagattagtgtgttttcaatttcttcagcaagcaacaatgaagctcgtcaggttcatgcgatatcttcatttttttgagtttcttttccagtgagcttagcaatggatttaactttacctttttgtgcagatttttgatgaagctgaacaacgagactgtctcaattgagctcaaaaacggcaccgttgttcatggaaccattacaggtcaccctccttatctttctttctttcttagctataatcatcaatgtaatttcttaagccaatatgtgaattatataatgtgtttgtatgaatttcaagttatttggaagtgtcgctaactgaactacaacaacaacataataattccacaagtggtgaggtcgtagagttaaagaggttctttccaatagattttcatgcttaaataaagcatttccaaacaattttataaaggatatacaaaaattagtgctcatgacagacaagtgaggttttgctcaagtggttgagcactggtaggttgagggtttgggtcagaaaaatagatcggaaactattgatccttctaggctcctgggggtggggtggaaaaacggagcagcaacaacaacgaaaccaagttgctaaactatctttggtttatgcctcaagttaagttagtttggtaactgcaatagtttacttcactctccttcacttccactgtgagtcactatgtatatccgtaaatgcattgaagctagggctgagaagctatagaaagtatatgtggtaactcgtctcaatctgtctactcctctcactgcccttctgaatttgttatcttcttatcctctctgattcacacgtattatctctacttcaatccttcaactatgaagttcctttgaaataccaataggaattcattctcaagtgcagagctatttctgcctatcatcactgtaatttatgtctagactctagatcactgtagttcatctctgtcaatgcatttactgtactctgggccaataaaaataacatcacttgttcgctttttttgtagctgtaaaagttggattattacacaactcgaagaaatttgaagtgtcactataataattcagcaattccttcagtgaattagtatgcatttaaatacaactgtgatgtctcaatcccaaactagttggtttgtctatatgagtgttctctatttgttctgctttattcggaccatttcattaattcacagttcgtatactgaatacaaatctctatattataaaacaaaacaaaaaactaagaaattatgggagtgaacatgggattcttatcatctttttgtcattttcttaatatttttcttaagttaaccccaatcatatattctttgctttgaaacttaaactatgtttggctggggtggaaaaaagaagcttaagagcaatggaagtaagaggttaagagttaatttcgttctaagtaaaaaaggcatataaagcaaatgtagatgggttgtgtatccttctatgtggatttttcagtatatctgaattttaatggaaaaaggaaatgtatctgtcttacttctgttccatccataccaaacaacctgaactacagcgcttctatccacttaagggaggaaaattactgaataaccaattcaccacataaggggaaagtaaattagtgaccaatgcttcacttaagggaggtagatcagtagaatctgtcttgacttaatattgagatagtagtattttctaatacttctaaattttcacgggttaatgtcaccattaatttgatacatcagtagaaagtataaatggtatttcttatactgctaatattttcaatgttgtgtaatattagcctgagataagtttaaacatagtaacatggtcaattggattcatttagtcgtaccgaacttttttgaaactgtggtgttgtttctttcgttgatgttgtaagaccttcattatcattttgtcaatagaataggtcgataatgtaatgatcttcagaaatttaagtctgcacatggctccacatcattatgccaccattgctctcaggtccctacaagtggaacattctatgctggaatcatttggtccaagagctctcttattttctatgcaaaaccatgttgtctttatccctttgtttccaaagatgagcattagttgctcatctcggcaaatgtaacactaattgacaaatattaaattttatctgtataaaattttgattacctagttgttgctatacgggtaacttataacacttgttagttgattatatgagcataaaccatgttgcctccacttgtttgtggattttcattgtctatgtgaggcgtgagtaagttcttatttcccagctcgagcttaacttccaggtacttaggtcagaaagcgtacgacacgtctagttctataggctggttgcatttgatatttgagcatgtagaactagcgcaaagagtgagaagtaagtggctatatgatgttgtgtactctgacttttgctgtgcaatgttaactaatcttaaattcaactagatgaggagctaaagctgtgtcgaactactgttattcactatgcatgaatacttgagcttatgctagcgtgcgatcaacttatagcttattatgttgtacccaagttttatgaaaacagcacctagtgttgcgtttataattcttatctgttgttgtaatcttgttgtgaatgttatctgtattcattgtactatagattttttacaattgaggagtagccatttatcccttatttctttttggtgctcccatgatatacctctatgcattgaagataggtgtcattcatagaatgatcgaacatcatatctttaggtaggcgttctgctttttggtatacatatacaggcgtgctttgtgcctacattttaaaaaataataattgctttatcctctaaaaaagatcatcatactgtagcttctttgtctggctgtagagatgccttagcaaacttagataggcttcttatggcaagtgccagggcttgtgctggttgcatcaaaaatgtcaaatttgcatggaaaatagctactatgtattgcagagtttatctctgttctagataactatagtagtcaaaagtagatattttgtcctgttctcatattttgaggcatagcattttcctttctctgttggttgtcctttccatgcttttgacttttattccttgaggggttgcttatacttacagaaaattgctagatataatagggatatagaaatttgtattcgagcatatcagtatgtgttttgatattaactaggcacagcggctatttcatctgtctccttattgaagttgctaaattgagagctgtgagttaaagtttctaaaggcagttacatttttacaggtgtggatgttagcatgaacacacatctgaaggctgtcaaaattacgctaaaaggaaagaatccagtgacattggatcacctgagtgtgaggggtaacaacatccgttattacatcctccctgacagcttaaatcttgagacgttactggtggaagaaacacctagggtgaagccaaagaagccaacagctggtattgttaaggactacgttcagatgtttttgtgcattgtcggtgtatggtaaaatttccttattacttggacatccttctaatacgctcttgctttgattgttccccttcaggaaagcctatgggacgtggtcgtgggcgcggtcgtgggcgtggacgtggtcgaggccgctaaatgcattccggctggtctatttttttgcagtcttgcctatgtaaacatgtgtacggattgatagatttgctgttagtttttatcctgtagaaaaaagatggatctcatgcaacaatttattttggatctttgaagtcaggagcatgttatctcgttgcatcatattgtctgctaacattaatttcgtttcccgttcaatttagtcaccttatgcatgttttgagagttgagtcacatttagtgtcgattgagaatttagctc

SEQ ID NO: 21 - DNA, 4172 bp, Solanum lycopersicum
tcttatattcttagcattaaatagtgtgagacgacggcccacgtttagtccaatggacgttcccagacgaggcatataataagacatcacttgtgtattttgaacaacccctggcgctccttgccctaaactaaagccctagattagtgtgttttcaatttcttcagcaagcaacaatgaagctcgtcaggttcatgcgatatcttcatttttttgagtttcttttccagtgagcttagcaatggatttaactttacctttttgtgcagatttttgatgaagctgaacaacgagattgtctcaattgagctcaaaaacggcaccgttgttcatggaaccattacaggtcaccctccttatctttctttctttcttagctataatcatcaatgtaatttcttaagccaatatgtgaattatataatgtgtttgtatgaatttcaagttatttggaagtgtcgctaactgaactacaacaacaacataataattccacaagtggtgaggtcgtagagttaaagaggttctttccaatagattttcatgcttaaataaagcatttccaaacaattttataaaggatatacaaaaattagtgctcatgacagacaagtgaggttttgctcaagtggttgagcactggtaggttgagggtttgggtcagaaaaatagatcggaaactattgatccttctaggctcctgggggtggggtggaaaaacggagcagcaacaacaacgaaaccaagttgctaaactatctttggtttatgcctcaagttaagttagtttggtaactgcaatagtttacttcactctccttcacttccactgtgagtcactatgtatatccgtaaatgcattgaagctagggctgagaagctatagaaagtatatgtggtaactcgtctcaatctgtctactcctctcactgcccttctgaatttgttatcttcttatcctctctgattcacacgtattatctctacttcaatccttcaactatgaagttcctttgaaataccaataggaattcattctcaagtgcagagctatttctgcctatcatcactgtaatttatgtctagactctagatcactgtagttcatctctgtcaatgcatttactgtactctgggccaataaaaataacatcacttgttcgctttttttgtagctgtaaaagttggattattacacaactcgaagaaatttgaagtgtcactataataattcagcaattccttcagtgaattagtatgcatttaaatacaactgtgatgtctcaatcccaaactagttggtttgtctatatgagtgttctctatttgttctgctttattcggaccatttcattaattcacagttcgtatactgaatacaaatctctatattataaaacaaaacaaaaaactaagaaattatgggagtgaacatgggattcttatcatctttttgtcattttcttaatatttttcttaagttaaccccaatcatatattctttgctttgaaacttaaactatgtttggctggggtggaaaaaagaagcttaagagcaatggaagtaagaggttaagagttaatttcgttctaagtaaaaaaggcatataaagcaaatgtagatgggttgtgtatccttctatgtggatttttcagtatatctgaattttaatggaaaaaggaaatgtatctgtcttacttctgttccatccataccaaacaacctgaactacagcgcttctatccacttaagggaggaaaattactgaataaccaattcaccacataaggggaaagtaaattagtgaccaatgcttcacttaagggaggtagatcagtagaatctgtcttgacttaatattgagatagtagtattttctaatacttctaaattttcacgggttaatgtcaccattaatttgatacatcagtagaaagtataaatggtatttcttatactgctaatattttcaatgttgtgtaatattagcctgagataagtttaaacatagtaacatggtcaattggattcatttagtcgtaccgaacttttttgaaactgtggtgttgtttctttcgttgatgttgtaagaccttcattatcattttgtcaatagaataggtcgataatgtaatgatcttcagaaatttaagtctgcacatggctccacatcattatgccaccattgctctcaggtccctacaagtggaacattctatgctggaatcatttggtccaagagctctcttattttctatgcaaaaccatgttgtctttatccctttgtttccaaagatgagcattagttgctcatctcggcaaatgtaacactaattgacaaatattaaattttatctgtataaaattttgattacctagttgttgctatacgggtaacttataacacttgttagttgattatatgagcataaaccatgttgcctccacttgtttgtggattttcattgtctatgtgaggcgtgagtaagttcttatttcccagctcgagcttaacttccaggtacttaggtcagaaagcgtacgacacgtctagttctataggctggttgcatttgatatttgagcatgtagaactagcgcaaagagtgagaagtaagtggctatatgatgttgtgtactctgacttttgctgtgcaatgttaactaatcttaaattcaactagatgaggagctaaagctgtgtcgaactactgttattcactatgcatgaatacttgagcttatgctagcgtgcgatcaacttatagcttattatgttgtacccaagttttatgaaaacagcacctagtgttgcgtttataattcttatctgttgttgtaatcttgttgtgaatgttatctgtattcattgtactatagattttttacaattgaggagtagccatttatcccttatttctttttggtgctcccatgatatacctctatgcattgaagataggtgtcattcatagaatgatcgaacatcatatctttaggtaggcgttctgctttttggtatacatatacaggcgtgctttgtgcctacattttaaaaaataataattgctttatcctctaaaaaagatcatcatactgtagcttctttgtctggctgtagagatgccttagcaaacttagataggcttcttatggcaagtgccagggcttgtgctggttgcatcaaaaatgtcaaatttgcatggaaaatagctactatgtattgcagagtttatctctgttctagataactatagtagtcaaaagtagatattttgtcctgttctcatattttgaggcatagcattttcctttctctgttggttgtcctttccatgcttttgacttttattccttgaggggttgcttatacttacagaaaattgctagatataatagggatatagaaatttgtattcgagcatatcagtatgtgttttgatattaactaggcacagcggctatttcatctgtctccttattgaagttgctaaattgagagctgtgagttaaagtttctaaaggcagttacatttttacaggtgtggatgttagcatgaacacacatctgaaggctgtcaaaattacgctaaaaggaaagaatccagtgacattggatcacctgagtgtgaggggtaacaacatccgttattacatcctccctgacagcttaaatcttgagacgttactggtggaagaaacacctagggtgaagccaaagaagccaacagctggtattgttaaggactacgttcagatgtttttgtgcattgtcggtgtatggtaaaatttccttattacttggacatccttctaatacgctcttgctttgattgttccccttcaggaaagcctatgggacgtggtcgtgggcgcggtcgtgggcgtggacgtggtcgaggccgctaaatgcattccggctggtctatttttttgcagtcttgcctatgtaaacatgtgtacggattgatagatttgctgttagtttttatcctgtagaaaaaagatggatctcatgcaacaatttattttggatctttgaagtcaggagcatgttatctcgttgcatcatattgtctgctaacattaatttcgtttcccgttcaatttagtcaccttatgcatgttttgagagttgagtcacatttagtgtcgattgagaatttagctc

SEQ ID NO: 22 - DNA, 20 bp, Artificial sequence
  Primer to amplify Solcy09g064660.2.1 gene region
attttgaacaacccctggcg

SEQ ID NO: 23 - DNA, 20 bp, Artificial sequence
  Primer to amplify Solcy09g064660.2.1 gene reg
actctacgacctcaccactt

SEQ ID NO: 24 - DNA, 2935 bp, Solanum lycopersicum
tccaatggatgaggcagcccatgtgctaatacgacgtcgttatagtgttttttgaataaccctggcgctctttgccctaaacttgaggtcgtgttcgatctgcgcttcgacttaagccctagcgtctcttcaatttcttcagcaacaatgaagctcgttaggttcatgcgatatcttcatcttttgagtttcttttccagtgtgcttagcaatggatttaacttttacctttttttgtgcagatttttgatgaagctgaacaacgagactgtctcaattgagctcaaaaacggcaccgttgttcatggaaccattacaggtcaccctccctatctttctgtctatttctttgttatctatatttaggtatttccctgtcaagtctaaaatgaacaatctgaacgtatgtaattgctgaattgctcgctccaataggtaaattaagaatgtgcttgtataaaattcatttcctgggttcacaagttttttggtagtgtcgctaactgaaagtctagttgctgggtggtatatggaagttgctgaactacaacaataacatactacccagtgtaattccacaagtggtgaggtcttgggagacagagtgtttgcaagccttacccctacctcgtagtgttaaagaggctgtttccaatagattctcatgtttaaataaaccatttccaaaccattttttaaataaaggatatataaaaataagtgctcatgacagacaagtgaggttttgctcaagtagttgagcacttccaccatcaactagtaggttgagggtttgggtcagaggatagatgggaacactattgatccttctagtctcatgggggtggggtggaaaaatggagcaacaacaacaacgaaagcaagttgctaaactgtctttggtttatgccacaagttaagttagttcatagcgaatgttaactgcaatagtttacttcactctccttcacttccactgtgagtaattatgtatgtccataaatgcatggagctaggactgagaggctatagaaagtatacgaggtaactcgtctcaatctgtctactcctctcactacccttctgaatatgtcatcttcttatcctctctgattcacacgtattatctctacttctgtcctccttcaactatgaagttcctttcaaataccaactggaattcattctcaagtgcagagctatttctgcctatcatcactgtaatttatgtctacactctagagcaccgtagttcctctcagttaatgcgttaattatactctgccaataagaataacatctctcattcactttttgtggctataaaaattggattattacacaactgaagaattttgaagtgtcactataataattcaacaattccttcaatgaatttgtatgcatttaaatacaactatgatgtctcaatcccaaactagttggtatgtctatattagtgtcctatatttgttctgctttatttggaccatttcattaattcacagttcgtttacttaaaatacaaatctctatattgaaaaacaaaacaaacaaaaaaaaaagaaattatgggagtgaacatgggattcttatcatctttttgtcattttcttaatatttttcataagttaactccatagatatattctttgccttaaaacttaaactatgtttgactttggtggaagtggcggcgtttgcgaatggctatgtttagcgtggggggtggaagtgatagtgtttgcgaatgagtgtgtttagcatgggggttgaagggtaagtcaggggtaaattagtaatttgaattttaagttgggagttgatgctttaaagtaatatagatagatatagacaactactttatcatctgcaagaaaatcatacagtagcttctttgtctggcagtagagatgccttagcaaacttagataggcttcttatggcaagttgcatcgaaaatatcaaattttgcaaggaaaatagccatatatgtattgcagagtttatctctgttctagataactgtagtagtcaaaagtaaaaatttgtcctgttctcatattttgagacctatagcatttctctttttctgttggttgtcctttccatgcttttgactgttattccttgaggggttgcttatacttgcagaaaattggtagatatattcgggatatagaaatatgtatttgagcatatcagtatgtgttttgatattaactaggcacagcggctattaaatctgtctccttattgaagttgctagattgagagctgtgagttatagcttctaaaggcagttacatttttacaggtgtggatgttagcatgaacacacatctgaaggctgtcaaaattacgctaaaagggaagaatccagtgacgctggatcacctgagtgtgaggggtaacaacatccgttattacatcctccctgacagcttaaatcttgagacactgctggtggaagaaacacctagggtgaagccaaagaagccaacagctggtattgtcatattgttaaggactatgttgggatttttttgtgccttgtgggtgtatggctaaatttccttattgcttggacatccttcttatatactcttcctttgattgttcctcttcaggaaagcctatgggacgtggtcgtgggcgcggtcgtgggcgtggacgtggtcgaggccgctaaatgcattccagctgctcttaatttttgcagtgttgcctatgtaaacatgtgtacggatggagagatttgctcttagtttttcccagtagaaaaaagatggatctcatgcacaatttattttggatctttgaagtcaggagcatgttatctcgttgcatcatattgtctgctaacgttacttttgtatcgtgtacaattatgttcttccttgtcaagttactcaactaaattggtcacttcagc

SEQ ID NO: 25 - DNA, 1860 bp, Arabidopsis thaliana
attgctgcttttcctcctcccgctcttgcttcttgccattgctgtttcgagctctcctcaaagccctaattttctcctcttcttcttcattctctcaccatgaagctcgtcaggtatggtttctactcggattctctttatttcttcggtttaattggaatttgcttcgaaattttgagatttctctttgttttcatgctcttcgatgtttgatttcgtatctggattttttcgcaggtttttgatgaaattgaacaacgaaacagtttcaatcgagcttaagaacggaaccattgttcatggaaccattactggtacgcttctcttaaaaacccagctctattcgctgtttagggttttgtgtaaaatctacgatgcttacctgtaatcgatggttactcgcgtattcacaaattctgatggtgtagtttgagtttagctagtgtctttcccctaataatgctttgttggtattaaccctagtgacactggtcaagtcagatgcagatgagtacactagattttacttgaatcgaggtttatgagatagtttagttcttcttcaaactttgatcacccatgaggtagtttactttctcctttgatggttgtaaccaatggagctctcgaatttactaattctgatgtgtaattctgttaatagacctagaaatgcattgtgggtgttgaatctctcttgtttgcaataagtgaatgataatggtggtctagtcagatggagatgatacattttattttgctttatagttgggtgtatgagaatgcttagttcttctgggagtgagtttagcacatgattatatatgagatagtttactttagtttccctctttatcattgtggacctctcttgttaacaaatcctgaggcttaattctgttattagtgatagtatctgaagaagttagagtttagctgagtcttaattatttttttacctcgaaatgctttgttggtaaccctcttgttagcaagaactgagtgatacttgtcgagtcagatgcagatgattagatttagcacatgaaagtcactcttatctctttccttgtaaagaaaatctctgatttttcacagaacagttaccgtgtggcttcttgtttcaacttctcttttagtctgattgatctaatacaagtggtctctgctgttaatgtttggtttggtttcctttttctgccatcatcttgtttagaatgcaattatcaatcaactcactgactggtactatctacttgtgatgacttaatgcaggtgtagatgttagcatgaacactcatttaaaagctgtgaaactcactctgaaagggaagaatccagttacattagaccacttaagtgtcaggggaaacaacattcgctattatattcttccggacagtttgaatctggagactttgctggttgaagacactccaagaatcaaacccaaaaagccaactgcaggtaacaaaacatttcttttttctgaactatgcctttgtttttgtttttgtttttgcttagactcatctttctattgttctctttggtatcaggtaagattccagcaggtcggggacgtggtcgtggaagaggtcgtggacgtggccgtggtggtcgttaagcattttgcagtaatcaaaagtctggtcctttttggtttgtaacagcaggattgtaaaagatctatcttgtagtgagatttggtgtcactaagacaaattcttgttatctctttaagttttcatgaatttgagatttgaaacgttattgccaaacatacatcactcaatatgttttattattgacttgatcactttattacatagtctgataaaacgtggggagttcgagaaa

SEQ ID NO: 26 - DNA, 1082 bp, Arabidopsis thaliana
acacctttcgctgtttcctcgtcaatttctttgtttggttgttctttcttcgcatccaaagatgaagctcgtcaggtactgtttcgttctcagttcttcttttctttagaaaattcatgttgttcttaatcttagttagctcctggcatggaatccattcccaatattgagcctgagctgatttgtgttatatggttttgattggatcgagtaggtttttgatgaagttaaacaatgaaactgtgtctatcgagctgaagaatgggactgtggtccatggaaccatcactggtaagttcttattgttagcttccggtttctttgtagctcttctcatcttttcttctctctcgatttctccgttgctaggtcttatacaagtcgcgatctgttcccttattttcaattggccctaatcgcgtccttgtgatcacatgaataggttgagtaaacacacagttagagccagtagtcataaaggtatatttattgaagtttaagcttgatgatttgggcaggtgttgatgtgagcatgaacactcacttgaagacagtgaagatgagcctgaaggggaaaaacccagtaacacttgatcatcttagcttgaggggtaataatattcgctactacattcttcctgatagcttgaacctcgagactttacttgtggaagacactcctagagttaaacctaagaagcctgttgctggtactgcttcattcatttttggtgtttgtaatattcgtcttcataagctaaaagacttctttatcagtccatttattcgttctctctttctttcttctttagggaaagctgttggacgcggtcgcggacgtggccgtgggcgtggtcgcggcagaggtcgttagatgtagcagcgctctggaacatgttgaaggtttgtgagtttaatttttaggagaatgtacgagttatgtaactagcaactctggcaaaggaaagatttgattgagaaatagtatgattgatgctctagtaaggagttactttgtaatggaattccatggataacaagatttggctttacatgttataagagcatctacaagtagagaa

SEQ ID NO: 27 - DNA, 2315 bp, Glycine max
agaagaagtaaagatagacaatagattgaaaagtggaaattcaaaattcaaaatcacaaaattgtcccttaaacccttacaaaagaaccgcttcttcttctgttcatctctcccactctcacttttaattttttgccctaaaaccctcgctaccgccgtggtggccgccgaaggtaagttttcatcttctctgatctgcatcgttgctattgccttctaaatttctgcattattattttaatttgtgcaaaagaaaatgttcacacccttgtttttgttttctctgtattgcttcacagaaggagagtagagagcgcaatcgagtcttcatcttcgactcaccatgaagctcgtcaggtttttctctttctttgttgcgttatctgttaattgcgtttttatttttttttcattttctgagatcaaagtctaataatatacgaacactgtatgagtaggtttttaatgaagttgaacaatgaaaccgtgtcaattgagctcaagaatggcactattgttcatggcaccattacaggtttcttttctttctctaattttttcccccagtgttgtacaagtgtagaaccatttacaagcaacatattagagttgaattcagtgtagtttcgagcttgattgtgaggcatttaattatttgcaagggttctctctactcttcaggtgattactgattagaatagattatacttcagtaccttcaactttttaggcattttcttggtctggtatgggtaaacatatataaaagaccatctcattgggtcgcgggtttctattagcatgaaagtggagagggattggggatttggcttccaaaattaattttctttggcagctgaataactacaaaggtttcctttactgagtagttcattctgggggtaatttggagtataattagactgtgctgctgtgtaggatgataattttgagatactgcaatttcttatagttcttataatggtatgagacaagctaactcagtgggctttggatgatggtttattcttgtattaagtttttggcagataatttgtggggaaatgatcctttcctggaagatctcaggacctcaatcacctgtctactaagcttctcactcaacaaatttctcttctcatggctcagtattttcttgaaatttcaacttttagaggaacttgcatgatagtgaaactactgaactagacttcctgaccatcttacactcttacaatttacagttttacttgattctagtaatatcttccctagttcaaatagatcaatttggattcccccccacccacacccactctaagatgtaactttaccattgtttgattgaaattattgcaatcaatctcttccgtgaaatttcctattggacacccatctgagttgagtttgatccagccacgtgggtgtgtagataataaatctttgatttagtcagtccttttactatcttgcttctttctagttgaactacaccagcaactttaagagaggaagtcttagcttactgattctgggacttttgccacaggtgttgatatcagtatgaacacacatttgaaaacagttaaactaactctgaaagggaaaaatccagtcactctagatcatctcagtgtgaggggtaacaacatccgttactatattcttcctgacagcttgaatcttgagaccttgctggtggaagagacacccaagatcaaacccaagaagccaactgctggtactgtttatagtttgactacaaaattttcatccatactcaattcactctaattgtttcttggtgagttccccaggttttaattttagcatattttcattacatttccaggcaaacctttgggacgtggacgaggaaggggccgtggccgtggtcgtggtcgtggtggtggccgttgatttgcacctttcttcatttttgtggcatgtggttgtaagctatgcagcaagaatttctgggtggttttaggaacttctacggagcatattttttattcctgctattttcttgcaagagcttatcatgctttagtgtacaaccatcttccttctgaaactgatgtaacatgaattacctatctgctattgccttatactagttgttaatggggatctattcattgagcagtatgacaaatcgcccacgactttggtcatacataacacagtaaataggcccaagagttgcctaatgagtttgaactagatgatggatttcctgatggcacatattgtgttatttatttatttacatgtatgaatggtatttcttaaacgtcttatttttgtttgct

SEQ ID NO: 28 - DNA, 10500 bp, Capsicum annuum
atggttttaacctttaactttgtgtgcagatttttaatgaagctgaacaacgagacggtctcaattgagctcaaaaatggcaccgttgttcatggaactattacaggtcactctctctctatctttctcggtatatattttggatagatacctgttctacacttacgtagtagctgaattgctcacgcctatatgtgaattaaagtatgtgcttgtatgaatatgattttctgggtggagaagttattttaaattgctgataaccgaaagtgtagttgttgggtgggatatgcaagttgctaaacttgatttgtaatgccacacgttaaattagttcatagaagatgaatggtaactgtaatagcttacttccagcgtgggtcactatgtatgtctataaaatgcatgaaagctatgtagagaagctatcaaaattatatgagttaacctgtctcgatctgtcttctcccctctccgttcttctgaatttatgaatcttttcccttcttatcctctctgatccgcacgttttgatacaagccaaacggccatcttagcctttgatgccatcgttcgaggccaacacatggaaaatctgattttggaaactttgaaggcccgagagtatgcacggggaagccattgtgggcactcaatagggcaatcccgtgtgtgtaaggttgcatgggggcttgccttgatgcccgagagctacaagtgttgaagtatcaaccatccacagttggagcattgaagaggtgtccattcattggtgtcttaacactccaagaccgccccttcattaagggtgtcttggtcattttatgtccctccttgactccataggtgcctctttcattaagggtatgttgttattttatcttcatcttaggcttatttttcagcttagttgtttattgatattgagagaactcatctcttgagagagtgttataaccgtaactagggtagcacttagtgtgaaatcactagtgttgcattttggctagaaatgttggtgcttgaggagagtgtgttccctcttgtgtctacgtaagagcttggtgttgattatatgagttgtaggggttttgacgtttgatacacgtcttggttcttaggttcttgtataattttaggtcaagttactatcctttatttattgcattgttgttgtgtttcgtatcttgtcttcattatctcgtttctttgtgttgctgcctcatttgttgttatcttgttttcagctttgttcttgtgttttgtggctgtttttagtgtcccgtactcgtctctgtatcatttggtatgagagcaatgattgattttgttccaataaaattaatcttgggcttgttaactcgaaaatcacaaaaaaaaagtgttgaaaaattgaaaatccaaaaagaaacattctgtctcgtttttagtctttggtcgaaattttgaacttagatttaggaggtttttatgtgttttggttgtttctagtgttagccccttcattactaacactaaaagagtctaaatctaagtttggagtcatttccatgttcttggaatttgaagattgttgttgttgtggatttgagttgaacgtgtgttgttattgaagatgtgggcttgaaaacgtgttgttgttatagagttcaacgtgagctaaaagagacaaaaatcattcaatttggagctcatttggtttggttttcattcttgaatgttcttgttgttcttgcttcttcatgttgtgttgaagaagaatcctcaaaggaggttgtttgatctaaaaatcaagtgaaagaaagattggtctttgtttgtcttggaaacattttcaagacaaaaaggtttgacttgttttgcacctttttggcaagacaacctttttgaggggtttgtctcctttcctcttttgtcaatcttgaaaaatgctctttctcctttttggcaataagtctttttgaaagtcttattgtccctttccttctttgtatacaaacaaagaccactctcttttcacttgattttcttgatcaaacaaccattcttgaaaatttctttccaacaagatatgtagatggtgaagaacacctcctttgaaattttggagttctaggtgcacattggacctccaaacatcaacaactatgcgttttcaagttcaaatcttcaacaacacattttcaagccaccaatcctcaaaaaacagcaactttcaaattccaagaacaactcgaaaatggctcaaaatttggatctagactcacttagtgttagtgaagaaggggctaatactagaaacaaccaaaagtccaaaacacataaaaatctcataaatctaagatacaaatttcagccaagacaccaaaaatgggacaatttttttttttttgagatttttagattttcaacacttgtttttttgtgattcgcaagttaacaagccccagattgatttcgtaggaacgaaatcaatccttgcgctgataccaaatgataacggatcgactacgaacacacaaaaacagccaagaacaacaaggaaaaccgagaacaagatacaacaaaaacaatggcagcagcaacaacacatgataatggaaacaagacaacaaatgcaataagataaagatagttagtttgatatagggagattacaaaacctaagaaccaagatgtgtttcaaacgtcaagacccctaaatcccgtaataactaacaccaaactcttatgtagactcaagagggacgtcaatctcctcaagcaccaacgtttctaagccaaaatgcaacacaagtgaatccacacttaatatgccctaattacggtttcactctctttctctctagagagaagacttgttctttcaataatccatatcaataatcaactaaggaaataaaccctaaaatgttctacttatagtctattacaaaagaagataaaatgatcaaactatccttaatgagaaaggggtgcctatggagtgtcaagacaccatgtgcaatctctaaattacccttaaagaagggccagccttggtgtgtaaattagggggctgccttggagtgtgtaaagctcttcaaaacacttcttcacttgtgctctcaggtagtcattaaggcaagcctccaagcaaccctccacacacggaattgctctcttgaatgttcaaaacgggtcctccatgcatgctcttgtgtcctcgaggtgatcaaagcttgagtttttaagtgttggcctccaaggatgtcatcaaaagctatgttggccgtttggctcgtatcatcttcatcccacttcaatcattcttcgactctggagctcctttcaaattccattaggaattcattcgcaagtgcagagctattctgcttgtcatccctgtaatttatgtctatagcattgtaattcctctctgttaatgtgttaattgcactctgtttcaatataaataatatctcttgttcagcttttgtggttctacaagttaaattgctacgcaatccgaagaaattgaagtgttctatattcactatatataacaattcaacaatttccttcataagaaattttttgccagtactatggacgcttattgtaagctctccaactctgaaataataccttttcaagaaacggatgcagggagacatggtcaggaaactcgagtcttcaatgaatctctacacatttatcccaaactagttggggtcacctataagagtatcctaatttgttgtgcttttttcagaccatttcattaattcacacttcatatactgatatacaaatctctaaattaaaaaacaaaaacatagaaattgtgggagcgaacataagattcttattggagtatctttttagtcattttcttaatatttttcataagtttaacctcaatccacatattatttgctttaaaacttaaaactatgttaggtttgtgttggaaaaaaaaacttaagaggaacggaagtagagaggaaaattaagagataattctttgcaagtaagcaggaataggaagcaaaagtggataggttttgtattcttatatctgaaatttttcagtgtatctgaaatttaacttaaaaaggaaatgtttgtcttctgttccaatcataccaaacaaccagaagtacaacgcttctctccacttaagggaggtaaatcagtgaataacccgtactctaatgaaggggaaagtaaatcagtaaatgaaaaatgcttcatttaagggaggtgaatccgtagaatctctctcaacttgagattgatatagtattttctaatacttctaagttttcacgggtaactgtcaccatattttatacatcagtagaaaatataaagattttttgtacctcttatattcttattattatctttatgttgtataatattagcctgagatgaatttaaatggagtaacatgatcaacgaggattcatatagccgagtcaaacttgtttggaactgaggtggtgtttcttctgtgtcgttgtaagaccttccccataattttgtcgataaaatgggttgataatgtaatgctcttcagaagtttaagttatcttgcacttggttccagatcattatgccaccattgttctcattccccacaagtgaaaagttctatgctggaagcatttggtccaagagctttcttaatttctatgcaagaccctactatcttcatccacttgtttccaagatgtgcattagttgctcttatcccacaaatatagcattaattggcaaaacctatgttaaatggtcaatttctctatataaaattctgattaccttgtttttgctatactgtaacttataatacttgtatagttgatcatataagcaagattccttcacttgtttgtggattgtccttatctatgtgaggtgtgagcaagctattatttcccagcttgaggttaaagttccaggtacttacggtcagaaagtcgacacatacaagtgtagttatataggctggtcgatatgatatttgggcatgtagaactatccaaaagagtgagaagtaagtggcatatatgatatcgtgtactccgacagtcacttttgtcatgcaatgttactgatcatagattcaactggacaagaagctaagttatgtccaactcctgttattcagtgagtatgtatgaatggtcgagcttaagcgggcacaaaatcaacttgtagctttacatgttgtaccctggttttatgacaacaactcttaatgttgtatttataattcttttctgttgtctaatcttgttgtcaatgctatttatgtttatcttactgtattttttgtacaactgaggagtagcctcttatcccttattttctttttggtgcacccattaggtacctttgtgcattgaggattgggtgtcattcatagaaaatcatatctttaggtaggctttctacattttggtatacttcttatatacggtcgtgctttgtaccgaaacatttttaaaattagtaattactttatcctccaaaaaaaacatctgtagcttctttatctggcagtagagatgctacttggcaaaattaggtaggcttcttatggaaagtgccagggcttgtgctggttgcaggatgcatctgaaatgtcattggccagaagggcagcaaagataagggaacaccctttgctgctagctttctccctttttcttttcaccgtccaaatccaaattgtggatccttatttgcatggatgcttgattgattgccatctctacagaaatttctttctgttttagataactagactccaacaacaacattcccaatgaaatttcactacaaaatataccacatatagagacctacatgcttccgacttttatttcgtgtacggttgcttatgctaacagaaaattgctagatatatttgggatatataaatttgtgttcgagcatcagtatgtgttttgatattaactaggcacaatggctattcaatccgtctccttactgactttactaaattgagagctgtgagttaatgtttctaaagcgagttatatttttaatatttataggtgtggatgtgagcatgaacacacacctgaaggctgtcaaaattacgctaaaggggaagaatccagtgacgttggatcacctgagcgtgaggggtaacaacatccgttattacatcctcccagacagcttaaatcttgagacgctgctggtggaagatacacccagggtgaagccaaagaagccaacagctggtatgttcagacgtttctttttctttttcctttgtaattgctaaaaaagtaatgttcagaagtatttttatgcctcctggctgtatggttaaatttccttgatgcttagacgtatactaatttcctctttctctgattcttctttctcaggaaagcctttgggacgtggtcgtgggcgcggccgtggacgtggacgtggtcgaggccgctaaatgcattccagctactcttatttttttttttgcagtggtgtctatgtaaacatatatgtatggatggagagacttgctcttagttttctagcagaaaagagattgatctaatgcaacaatttattccggatctttaaagtctgagcacgttatttcgtttcgcacgatatatggtgtactaacatttctttcgtatctcattcaactttattctgcctattcatatcactgaactgtgttggtcacttttgcatctgcccatttgcattgtgcccttcggttttttcatacaaagtagttgttacttttaagctgcttactggcttaaccctatgaaactctaataatgcccacatttacttagtttagtctataataggaagtagtttgttaaaaattaaaatgacagaagatagaaaaagggaaaaagaagcaagatactactacgaactagtgttttgagtctctcacattaccagcctacatttggatcaaattgggaattggaggaatcattatgtgggatgatcgatacaagtttgaacccgggcacttttcatggatttgaaactccatcaaatttttaactaaaaattatgttgttagtttcatttaagatgggatttatgttgtttatacaatttttaaatcaactgaatcgtttcattttccttaagtttgccaaactggctaaaagttgaatactctactctaaaatattggatacaacttaaatagcaggctaacatagtgcaattgttaccattaaaaagtgacccggcccacatttagtcgattgggcctttttacatcgcccaaaatgaataggcccatgtgctacgggcacatggagtacgacgccgcttctgttgggagtctctggtttttgaataaaccctggcgctccttgccctaaacgggaggctgtatcgggagtatcttcaatttctccagcaagaatgaagctcgtcaggttcactcgaaatcttcaattattttttttcacagtgagtttagccattgattaaactttcaactttgtgtgcagatttttgatgaagctcaacaacgagactgtctcaattgagctcaaaaacggcaccgctgttcatggaaccattacaggtcactctctctctatctctatctttctcacattttattttgcatttttattctaatatatattttgtatagctgtatttacgtatttggtgtgacccttgtaaggctgtataatccgaagtgaataatttgcacttatgtaatagctgaatttctcatacagtcacaccaatatgtaaattaaagaatgcgcttgtatggatatgattttctgggtagataagttatttcaaattgttgctaactgaaagtttagttgttctgaaaaaaggggagtcaacatatattatatttacataaaactaaaatttttgacctagctaaacagtgaagttttccgtcgaaggggtatcaaatgacacccctcgccataaggtggctctgccactggtaagtggtaactgcaatagcttacttcattctcctttcacttccaccagtcactatgtaagtctgtaaatgcatggaatctagggtagagaagctataaagagtatatgaggtaatccgtctgatatcaatcggtcttcccctctctctgttcttctgaatttccgaatcttctgaatttccgaattcctttcttatcctctttgatttgcacattttatctccacttcaatccttcttcaactatgaagttcgtttgaagtaccaataggaatttattaagaagggacaggaagcaaaagtagatgggttgtctatccttctatctggattttttagggtaactggaatttgatggaagaagaaaatgtttttgtcttacttctgtccaaccttaccaaacaaacagaagtacatcttttctccacttacgggaggtaaatcactgaatggacatgctccacttatggtggaagtatacagtaagtggccaatgcttctctcattctctgtcaaataaaaagggaccaatgcttcgctttagggaggtagatcagtagaatctctctcaacttgagattgagatacaataatttatagtacttctaaattttaacgggtaactatcaccattacagtatatagcagtaggaaatatgaagactttttggtactgtttatggagtaacatggtccatgaggattcatttagccgacacgaacttgtttggaactgaggtgtcgtttcttttgttgttgttgtaaaaccttcatcatcattttgtctgataaaattggtcgataatgaaattctcatgagaaatttaagttatcttgcgcatggttcctgattattatgccaattgccaccattgttcttagttccctacaagcgacaagttcttttatgccggaattatttggtccaagagctttcttattttctatgcagaaccctgttgtcttcatcctgtcgtctccaagacgtgtgttagctgctctaatccccacaaatgtaacattaattgacaaaacgtatgttcagtgatcaatttcatctgtctacattttgattacctagttgttgctacaccgtaacctatgccacttgtaatcgattatgtaagcaataaccatgatggcttcacttatttatggattgtctatgtgaggcatttgtaagctactatatcccagcttcatgttaaagttccagcctccaggtatttgctatatttaatgtttcttgttcttagatcaactggacaaggagctaagactttttttttttttttggggataaccgtggtgtccgggccatcttgcccgcacctcgactaatttcacgggatacttgccacctcccaccagtcaccggtcaccaacaacatgtaccaggtaactttgtccaccaagactagaacaaatggaaagaaatcacctagtgtttgtctctgttgggaattgaacttgagacctcatggtgctcaacccaacttcattgaaccactaggccacaactttgggtgcaacacgtagcgaagagttgtgtgcaactcttgttattcaccaagtatgtttgaatgcttgagcttaagtgggtgtgaaatcaacttatagcttattatgttgtactcaagttttacgacaacaccacttagtgttgcaagtataaggaaatacgagcacgtttcgttgtgaaaagaagagaggtaatagaagaaccaataattgttaccctgcaaaacaggacatggaccatctaatttatgggtcctaattttgcgtggaggcttagttgatggccatatatgtattccagaagttttatctcttttctaaacgattagaatagttaaaacataaatttttgtcatatcctcctatttagagaccttttgcattttttttctctgttggttgtcctttctatgcttccgacttttattccgtgaagggttgcttatactaacagaaaattgcttgatatatttgggacacattaatatgtcttaagcatatcagtatgtgtttcgatgctaactaggcacaatgtctattacatccgtctccttactatttactaaattgagagctatgagtttagtttctaaagggagttatatttttataggtgtggatgttagcatgaacacacacctgaaggctgtcaaaattatgcttaagggaaagaatccagtgactttggatcacctgagtgtgaggggtaacaacatccgttattacattctccctgacagcttaaatcttgagacgctgctggtggaagatacacctagggtgaagccaaagaagccaacagctggtattgtcatattcttaagggctatgttcagattgtttttgcgccttctggctgtatggtttaatttccttgatgcttggacatccttctaatatcctcttcatttgtttcttcctcttcagggaagcctttgggacgtggtcgtgggcgcggtcgtgggcgtggacgtggtcgaggccgttaa

SEQ ID NO: 29 - DNA, 6572 bp, Capsicum annuum
atgaagctcgtcaggtttatgcaatttcttcaatttttttttgtctctgtgagtttttttttccctagtgtgtgtagccatggttttaacctttaactttgtgtgcagatttttaatgaagctgaacaacgagacggtctcaattgagctcaaaaatggcaccgttgttcatggaactattacaggtcactctctctctatctttctcggtatatattttggatagatacctgtaaaatctaagatgaacagtctacacttacgtagtagctgaattgctcacgcctatatgtgaattaaagtatgtgcttgtatgaatatgattttctgggtggagaagttattttaaattgctgataaccgaaagtgtagttgttgggtgggatatgcaagttgctaaacttgatttgtaatgccacactttaaattagttcatagaagatgaatggtaactgtaatagcttacttccagcgtgggtcactatgtatgtctataaaatgcatgaaagctatgtagagaagctatcaaaattatatgagttaacctgtctcgatctgtcttctcccctctccgttcttctgaatttatgaatcttttcccttcttatcctctctgatccgcacgttttgatacaagccaaacggccatcttagcctttgatgccatcgttcgaggccaacacatggaaaatctgattttggaaactttgaaggcccgagagtatgcacggggaagccgttgtgggcactcaatagggcaatcccgtgtgtgtaaggttgcatgggggcttgccttgatgcccgagagctacaagtgttgaagtatcaaccatccacagttggagcattgaagaggtgtccattcattggtgtcttaacactccaagaccgccccttcattaagggtgtgttggtcattttatgtccctccttgactccataggtgcctctttcattaagggtatgttgttattttatcttcatcttaggcttatttttcagcttagttgtttattgatattgagagaactcatctcttgagagagtgttataaccgtaactagggtagcacttagtgtgaaatcactagtgttgcattttggctagaaatattggtgtttgaggagagtgtgttccctcttgtgtctacgtaagagcttggtgttgattatatgagttgtaggggttttgacgtttgatacacgtcttggttcttaggttcttgtataattttaggtcaagttactatcctttatttattgcattgttgttgtgtttcgtatcttgtcttcattatctcgtttctttgtgttgctgcctcatttgttgttatcttgttttcagctttgttcttgtgtttcgtggctgtttttagtgtcccgtactcgtctctgtatcatttggtatgagagcaatgattgattttgttccaataaaattaatcttgggcttgttaactcgaaaatcacaaaaaaaaagtgttgaaaaattgaaaatccaaaaagaaacattctgtctcgtttttagtctttggtcgaaattttgaacttagatttaggaggtttttatgtgttttggttgtttctagtgttagccccttcattactaacactaaaagagtctaaatctaagtttggagtcatttccatgttcttggaatttgaagattgttgttgttgtggatttgagttgaacgtgtgttgttattgaagatgtgggcttgaaaacgtgttgttgttatagagttcaacgtgagctaaaagagacaaaaatcattcaatttggagctcatttggtttggttttcattcttgaatgttcttgttgttcttgcttcttcatgttgtgttgaagaagaatcctcaaaggaggttgtttgatctaaaaatcaagtgaaagaaagattggtctttgtttgtcttggaaacattttcaagacaaaaaggtttgacttgttttgcacctttttggcaagacaacctttttgaggggtttgtctcctttcctcttttgtcaaccttgaaaaatgctctttctcctttttggcaataagtctttttgaaagtcttattgtccctttccttctttgtatacaaacaaagaccactctcttttcacttgattttcttgatcaaacaaccattcttgaaaatttctttccaacaagatatgtagatggtgaagaacacctcctttgaaattttggagttctaggtgcacattggacctccaaacatcaacaactatgcgttttcaagttcaaatcttcaacaacacattttcaagccaccaatcctcaaaaaacagcaactttcaaattccaagaacaactcgaaaatggctcaaaatttggatctagactcacttagtgttagtgaagaaggggctagtactagaaacaaccaaaagtccaaaacacataaaaatctcataaatctaagatacaaatttcagccaagacaccaaaaatgggacaattttttttttttttgagatttttagattttcaacacttgtttttttgtgattcgcaagttaacaagccccagattgatttcgtaggaacgaaatcaatccttgcgctgataccaaatgataacggatcgactacgaacacacaaaaacagccaagaacaacaaggaaaaccgagaacaagatacaacaaaaacaatggcagcaacaacaacacatgataatggaaacaagacaacaaatgcaataagataaagatagttagtttgatatagggagattacaaaacctaagaaccaagatgtgtttcaaacgtcaagacccctaaatcccgtaataactaacaccaaactcttatgtagactcaagagggacgtcaatctcctcaagcaccaacgtttctaagccaaaatgcaacacaagtgaatccacacttaatatgccctaattacggtttcactctctttctctctagagagaagacttgttctttcaataatccatatcaataatcaactaaggaaataaaccctaaaatgttctacttatagtctattacaaaagaagataaaatgatcaaactatccttaatgagaaaggggtgcctatggagtgtcaagacaccatgtgcaatctctaaattacccttaaagaagggccagccttggtgtgtaaattagggggctgccttggagtgtgtaaagctcttcaaaacacttcttcacttgtgctctcaggtagtcattaaggcaagcctccaagcaaccctccacacacggaattgctctcttgaatgttcaaaacgggtcctccatgcatgctcttgtgtcctcgaggtgatcaaagcttgagtttttaagtgttggcctccaaggatgtcatcaaaagctatgttggccgtttggatcgtatcatcttcatcccacttcaatcattcttcgactctggagctcctttcaaattccattaggaattcattcgcaagtgcagagctattctgcttgtcatccctgtaatttatgtctatagcattgtaattcctctctgttaatgtgttaattgcactctgtttcaatataaataatatctcttgttcaccttttgtggttctacaagttaaattgctacacaatccgaagaaattgaagtgttctatattcactatatataacaattcaacaatttccttcataagaaattttttgccagtactatggacgcttattgtaagctctccaactctgaaataataccttttcaagaaacggatgcagggagacatggtcaggaaactcgagtcttcaatgaatctctacacatttatcccaaactagttggggtcacctataagagtatcctaatttgttgtgcttttttcagaccatttcattaattcacacttcatatactgatatacaaatctctaaattaaaaaacaaaaacatagaaattgtgggagcgaacataagattcttattggagtatctttttagtcattttcttaatatttttgataagtttaacctcaatccacatattatttgctttaaaacttaaaactatgttaggtttgtgttggaaaaaaaaacttaagaggaacggaagtagagaggaaaattaagagataattctttgcaagtaagcaggaataggaagcaaaagtggataggttttgtattcttatatctgaaatttttcagtgtatctgaaatttaacttaaaaaggaaatgtttgtcttctgttccaatcataccaaacaaccagaagtacaacgcttctctccacttaagggaggtaaatcagtgaataacccgtactctaatgaaggggaaagtaaatcagtaaatgaaaaatgcttcatttaagggaggtgaatccgtagaatctctctcaacttgagattgatatagtattttctaatacttctaagttttcacgggtaactgtcaccatattttatacatcagtagaaaatataaagattttttgtacctcttatattcttattattatctttatgttgtataatattagcctgagatgaatttaaatggagtaacatgatcaacgaggattcatatagccgagtcaaacttgtttggaactgaggtggtgtttcttctgtgtcgttgtaagaccttccccataattttgtcgataaaatgggttgataatgtaatgctcttcagaagtttaagttatcttgcacttggttccagatcattatgccaccattgttctcattccccacaagtgaaaagttctatgctggaagcatttggtccaagagctttcttaatttctatgcaagaccctactatcttcatccacttgtttccaagatgtgcattagttgctcttatcccacaaatatagcattaattggcaaaacctatgttaaatggtcaatttctctatataaaattctgattaccttgtttttgctatactgtaacttataatacttgtatagttgatcatataagcaagattccttcacttgtttgtggattgtccttatctatgtgaggtgtgagcaagctattatttcccagcttgaggttaaagttccaggtacttacggtcagaaagtcgacacatcaagtgtagttatataggctggtcgatatgatatttgggcatgtagaactatccaaaagagtgagaagtaagtggcatatatgatatcgtgtactccgacagtcacttttgtcatgcaatgttactgatcatagattcaactggacaagaagctaagttatgtccaactcctgttattcagtgagtatgtatgaatggtcgagcttaagcgggcacaaaatcaacttgtagctttacatgttgtaccctggttttatgacaacaactcttaatgttgtatttataattcttttctgttgtctaatcttgttgtcaatgctatttatgtttatcttactgtattttttgtacaactgaggagtagcctcttatcccttattttctttttggtgcacccattaggtacctttgtgcattgaggattgggtgtcattcatagaaaatcatatctttaggtaggctttctacattttggtatacttcttatatacggtcgtgctttgtaccgaaacatttttaaaattagtaattactttatcctccaaaaaaaacatctgtagcttctttatctggcagtagagatgctacttggcaaaattaggtaggcttcttatggaaagtgccagggcttgtgctggttgcaggatgcatctgaaatgtcattggccagaagggcagcaaagataagggaacaccctttgctgctagctttctccctttttcttttcaccgtccaaatccaaattgtggatccttatttgcatggatgcttaattgattgccatctctacagaaatttctttctgttttagataactagactccaacaacaacattcccaatgaaatttcactacaaaatataccacatatagagacctacatgcttccgacttttatttcgtgtacggttgcttatgctaacagaaaattgctagatatatttgggatatataaatttgtgttcgagcatcagtatgtgttttgatattaactaggcacaatggctattcaatccgtctccttactgactttactaaattgagagctgtgagttaatgtttctaaagcgagttatatttttaatatttataggtgtggatgtgagcatgaacacacacctgaaggctgtcaaaattacgctaaaggggaagaatccagtgacgttggatcacctgagcgtgaggggtaacaacatccgttattacatcctcccagacagcttaaatcttgagacgctgctggtggaagatacacccagggtgaagccaaagaagccaacagctggtatgttcagacgtttctttttctttttcctttgtaattgctaaaaaagtaatgttcagaagtatttttatgcctcctggctgtatggttaaatttccttgatgcttagacgtatactaatttcctctttctctgattcttctttctcaggaaagcctttgggacgtggtcgtgggcgcggccgtggacgtggacgtggtcgaggccgctaa

SEQ ID NO: 30 - DNA, 2367 bp, Cucurbita moschata
gtaatggttgtacctgtacgcgccagctgcagtattttctcataccaagcaccccacttctttatttgtttatttattatacgtaattttctttttcgtccttttattctttatattatcatttatatccacttattttctccatttacttatcaggcgtaccacatacttgaaattgtacaaatttcccctcccgcctcctttgtttgtccgtgcttgaaaccctacattttgcaattcccccgccatcatgaagcttgttaggtatgctcttcttttttcttactctgagcttcttttttctcgaactactcgttgtttgatgtatttagtggtcgttttatgtttagaaacttggttttgatgaattcacaggtttttgatgaagctcaacaatgagacagtttcaatcgagctgaaaaatggaaccgttgtccatggcaccatcacaggtttccatttctctttcactcactaactcgcactgcataaaaacttcattaatcttgttaaacccttaaaccctagatttacgaacttactggcagaggttttttaaactttaacttccattctgttttcatctttgtacgccactatattggaagttgtagaatatctcagtgcaaagtgataattggaatttggattcttttgtcaatttagagtggtttatgatataccacaagccttctggctcttgataaccttggaatacgtagatgtttctcggcgacattgctacaaactgggggtgacaacccaacttgggaactttattttagcttctcttggggtgttgaggcttctgtaagtaagagactgcacaaacatcctgtctatcctggggagattcataacttattttaaccaaaggacttttcagctttagcaggttttggatagttcaatgaaagtggttttagtggagatacagttggatgaatcaatggatcctactactttgtcttggccagcttctaattgaaccctattgatcttttctgcaaaggcaatccaactaccaatttctctatttaaaagagccaacaagcactccacaattatcctccaatgtaacctttggtttaggtttcatagcaaattcttgtggttccaccccatatgccgaccatcacctctcaccgtgaccatttaaggagggcactatttctctacttatagaagagccaaaccccaagacccaatggaagggaagtccaactccatttcgatcaagtatcttatatcccccatccccacaccccaaaaaaaaaaaaaaaaagttggtgattatgatgagattgcattttgttgtggacacctcctatccagcttccacttgatgtatttttctatgtgctgctggttctgaaactaattattgttgtcttttcttacaactgtttttatggaagagttctactcaatgtcatagttttagctggtcacaccttctgttgtaggatttcacttgaaactataaataagtgtcaatatatacaatatgcttgaataacataccttgcagtgcattatgacttggcctaattgaataagggtcatgtaaataacaaatgacttggagagaatggattcaagccatgttggttacctacttaggatttattatcctatgacttaccttggcaacagaatatagtagggtcagtcagttgtcgcaggttggatctgaaatgaatggatatgaaaaaaaatgaaaaataaaaacctatcttgcacttacagcttgtgaaatttgtttggcctaatatgttgaagcatctaaattacgctctacgttcctgttttctataatttatttctaaaacatgctcagatgagtattattattattttttttttaaaaaagaacaatgacggcatgaacttgatgtagaagctcttactctttttgcttaaaaccaagcattggtcaaggttagaagtcattgacgggctgattttcttgtatcacttaggtgtggatatcagcatgaatacacatttgaaggctgtaaagcttactctaaaggggaaaaatccagttaccatggatcatttaagtgtgaggggaaacaacatcagatattatattctacctgacagcttgaatcttgagactttacttgttgaagagacacccagggtcaagcccaagaaaccaactgcaggtatgcgctgtctttgcattttgcctgtactttatggtgttttctgaaagtcaaattgtttatactgaatcgtatgccacccagtgtcaaatatgttgcttatgtatttccccattcttttgcaactatagggaagcctttggggcgtggacgaggacgaggccgtgggcgtggtcgtggtcgtggacgctag

SEQ ID NO: 31 - DNA, 3599 bp, Cucumis melo
cttcattttcttcaatctattagaaacaaagaaagaaagaaaggaaagaaatagggacaaccgcagcaatctcccaatttcctctcccccgcttctgttgtttgaaaccctacattcttcctttcctctccaatcatgaagcttgttaggtatcttcttctctttttctatttttgccctctttttttcctcgatctacatccaaatgacgtttttcgttttcaaaatcttggttttcatacaggtttttgatgaagctcaacaatgagactgtctcaatcgagctcaaaaatggaactgttgtccatggaactatcacaggtctttctacctctctttcattaaatcttcttaaaccctagatttaccaactttgcggctgaggggtttttaacttccaacttctcttctattttcttctttgaacttcactgtattggaatttgtacaacatttcagtatgttcccccagtgtacaactctcactactattgctttcgtctcatttccatttcccgaccttttaaagctaactaaaatgttctctatcccgctcccatttttaatttaaaaaattagtattcaactcatgggttgcttaggagaaattaagttccttgtcttctggacgtttggaggatgagatgtttttctctgtattgtttttatttttattattttcataataatttgaacttttgattactgtctctactgatgctgaataagcattctatttcatgacatatctgtagatttaaacaaatgtttaaaatatcctacaatttattctttcccgtttgatttgatggaattgtttcgtgaatttacttcttctttctttttttttttttttttttttgaaatggaaacaaaacaagtatttatacttatacaatcatcttgaactgtttctcacctgcactagaatgatgttacgttagtctgattaaacatgaacttctgctgaatcccagcatttttggactcggcagcccatatgccattctttggttaattatatgtgtgtgtttttaattttgttttgcataagagactgcgcaataactgaaagagaagcaacttgtaacttgaactgttcccagtggtagaaacatagtcaattgagcagtagcaaattatctttgtttgtagcctttgtcctcctacactgcctctaacaacttgaactattcttaaccttccctggaatagcaataacttaatcttgattaatcaaaacttcttctgaactccattatttttgggttccgtatcccattggccattcttatatgaatttttacccttttttatcttttggtgagagactatgttacaagtgaagcaaaaattctaacctagggtaatcttcgtgctggtggagaaaacctgggagctataaaaaaaagagccttccgataaaagttaaatcatgttcataagtagactgtaattacaacacattatcttctatttgaaaaaccatcgtaaaaatgtgtatggtacactattaaaaatatattctaaaatagatatttttcctctcaaaatattcgtagaagtagtagtgcagattttatcctacatctaacccatcaggctgtgaatgttttaaaaaataatcgtcacccaagctagttaggcaaaagataaaccagttttgttgaaaatgaaaaacgatgctctatagacaaaaggacagtggaggtaaaggtaattttgggattccccatttgaataatagaggaacaaaccaggggagacaacccagcttgggaactttttttagattctcttggggggttgaggcttctattagtagccaacctcacaaacatcttaaccttcctaggggtgcccaacatccaaactgatttgactttacaaatgcatattttgctttagcaaggtctcagagagtttagtgaaggtgatcttagaggagaaacaatttaatgaatcaatgaattttacctctatgtcttggccagcttctgattgaaaagtatcgatctttcctataaaggctatccaactttccaatttctctgtgagaagagccaacaagcattccacaattattccccaagactatccatggttttggtttcatcgtcgtttctggttctagatggcaaccatcactttttgcaactatttaagaagtagcgcattatttctttacttactaaaagagccaaccaatggaaaggaagtccagctccattttaatgggtgacttaggattcgatgaattatgattagtttgcattttgttgttgacaaccccctatacatcttccactcaatgtattttgctatgctgctgctactgaaactagttatttaccggcagagttttcacgactcattattttagttggttacctcttttgttggaggatttcagttgagattgtacgcaatgttttaaaaggctaaaggcagcctcttccaaagttgtaagtctctttggttaaaaatactttaccaagtattgctgggcaacacttgataatcaggtaaaagtaaatataaaaaaaaaaaggaaaaatcactcttcccaatttccacctagacaaaaattctaaaatgatatctttaactttactaatattatttgattgacttatcccagaaatattatatcgtgacaaactaatattattcaataaactattacaaagaggtgtaagtctatgcatgcaatatgcttgataacctacattgcactttaccttgtgaaatcttgttttcgactatgatacattaatcaatgatttgttttaaaaaactcgatgaaatttcatgaacttaaagtagaagtacgcactcttttctcttaaaacctaagtattggtcaaggttagaagtcgctgatgggttgattttgagtggttttcttgtattacttaggtgtcgatatcagcatgaatacgcatttgaaggctgtgaagcttactctaaagggaaaaaatccagttaccatggatcatttaagtgtgagaggtaacaacatcagatattatatcctacctgacagcttgaatcttgaaactttacttgttgaagagacacccagggtcaagcccaagaaaccaactgcaggtttgtgttcttgcattctggcctgtactttatggtcattttctgtggcaagtccaattgtttatactgaatcatatcatttgctactcattatcaaatgtattgcttatgcatttctacactctactccaactacaggaaggcctttgggacgtggacgagggcgcggccgtgggcgtggtcgtggacgtggacgctagatttgatgcagtttcaatttttgagacctgtagtgtaaacacttgcattgtagggaaagattatgaattttgggacacgaaaaagtggtattgcaattcactgttcaatctcgtatgtagtttattacaatgctattcggttgtttttcatatgatagatgtagccttgctatgtttcctggtgcttgggatctgttaatggtttgctctaatatcatcacaatgataaatcgagtatgtctctatctcgttttttacttttaatcccaatagatttggtagtcatgc

SEQ ID NO: 32 - DNA, 3302 bp, Cucumis sativus
cctctcccccgcttcgttgtttgaaaccctacattcttcctttcctctccaatcatgaagcttgttaggtatcttcttctctttttctatttttgccctctttttcttctcgatctacatccaaatgccgtttttcatttctgaaatcttcgttttcatacaggtttttgatgaagctcaacaatgagactgtctctatcgagctcaaaaatggaactgttgtccatggaaccatcacaggtctttctgcttctctttctttaaatcttgttaaaccctagatttaccaactttgcggctcagggttctttaacttccaacttctgttctattttcttctttgaacttcactgcgttggaatttgtacaacatttcagtatgttcaccagtgtacgactctcactactattgctttcgtctcacttccatctcccgacctttgaaagctaacggaagggttctttatcccgctcccatttttaattttaaaaattagtattcaacttatgagttgcttacggcaaattaaattccttgtcttctggacgttgggaggatgagatgtttttctctctattgtttttatttttattattttcattttcaaaataatttgaacttttgattactctctactgatgctgaatatgcattctatttcatgatatatttgtagatttaaacaaatgtttaaaatgtcccgtcattttttcatggaatgactcctacaattcattctttcccatttgatttgatggaattgttttttcaaccaagtgaatatactttttttttttttgaaacggaaacaaaccaagtgaatatacttctacaattaacttgaggtgagaaacctgcactagaatgatgttactatagtctgattaagcatgaacttctgctgaaccccagcatttttggactcggtagcccatatgccattctttggttaattgtatgtgtgtttttaaatttgttttgcataagagactatgcaataactgaaagagaagcaacttgtaactgaactgtgcccagtggtggaaacatagtcaattgagcagtcgcaaattatatgcgcctttgtcctcctacactgcctctaactacatgaactatttttaaccttccctggaatagcaataacttagttttgattaatcttcttctgagttccattagttttgggttctgtagaccattagccattcttacatgaatttttacccttttttatcttctgatgagagactatgttacaagtgaagcaaaaagtctaacctaggtttaatcttggggggagaaaaccctaggagctataaaaaaaagagccttctgataaaagttaaatcatattcattagtagactgtaactacaacagattatcttctatttgaaaaaccatcgtaaaaatgtgtatggtacactattaaaaatagaatctaagatagatactttttctctcaaaatatttatagaagtagtagtgcagattttattttacatccaactcatcaggctgtgaatgttttgaaaaataattgtaacccaagctagttaggcaaaagataaaccagtttgttgaaaatgaaagaccatcctctatagacaaaaggatagtggagataaaggtaattttgggattacccattggaataatagaggaacaaaccaggggagacaacccagtttgggaatttttttctagattctcttggggggttgaggtttctattagtagccaacctcacaaacatgttaaccttcctaggggtgtcaaacatccaaactgatttgactttacaaaggcgttttcagctttagcaaggtctgagagagtttagtgaaggtgatcttagaggacaaacaatttaatgaatcaatggattctacttctatgtcttggtcagcttctaattgaaaggtatcgaactttcctataaagtcaatccaactttccaatttccctgtgaaaagagccaacaagcattccacagttatcccccaagactacctatggttttggtttcattgtcgttgtttctggttctagatgatcacttttcacaactgtttaagaagtagcacatcatttctctacttacaaaaagagctgaccaatggaagggaagtccgactctattttaatgggtgacttactgatttggtgaattatgactagtttgcattttgctgttgacaaccccctatacatcttccacttaatgtatttttctacgctgctggtactgaaactagttatttaccggcagagtttttttttcacaaggagttgtaacagaagagttgagactcttgtcataacttatgactcataattttagttggttacctcttttgttggaggatttcagttgagattatacgcaatgttttaaaaggctaaaggcagcctcttccagcatagccccaaggtgaggtgttatgagttgtaagtctctttggttaaaaatattttaccaaatattgttaggcaacacttaataatcatgtaaaagttaataaaaagaaaaaaggaaaatcactcgtcccaatttccacctagacagaagttctaaaatgatatctttaactttactaatattatttgattgacttctcctagaaatattagattgcgacaaactaatattattcaataaaattattacaaagaggtgtaagtctctatctatgcaatatgcttgatgacctacattgcactttgcgttgtgaaatcttgttttcgactatgatatgttaatcaatgatttgttttaaaaaaattcaatgacatttcatgaacttaaagtagaagtacgcactcttttctcttaaaacctaagtattggtcaaggttagaagtcgctgatgggttgattttgagtgtttttcttgtattacttaggtgtggatatcagcatgaatacgcatttgaaggctgtgaagctaactctaaagggaaaaaatccagttaccatggatcatttaagtgtgagaggtaacaacatcagatattatatcctacctgacagcttgaatcttgaaactttacttgttgaagagacacccagggtcaagcccaagaaaccaactgcaggtttgtgttcttgcattctggcctgtactttatggtcattttctgctgcaagtccaattgtttatactgaatcatatcatttgctactcattatcaaatgtattgcttatgcatttctacactatgctccaactacagggaggcctttggggcggggacgagggcgcggccgtggacgtggtcgtggacgtggacgctag

SEQ ID NO: 33 - DNA, 3151 bp, Citrillus lanatus
atgaagctcaacaatgagactgtctcaatcgagctcaaaaatggaactgttgtccacggcactatcacaggtctctctaactctctttcttaaatcttgttaaaccctagatttaccaactttgcggccgtggtttttctcccaacttccaacttccaacttccaacttccaacttccattccattttcttctttgaacttcactgtattagaagttgtagaatatttcagtatgtccgccagtgtacaactctcactattctttactttcgtctcatgaccatctgtgaacgtttgaaagctaactgaagggttctggcaagcgtgcacttggagaaccaccttcccactcccatttttaagtttaaaaattagtattatcatcaattcttttaaaagtaaattagtattgaactcatgggttgcttacgtgaaattaaatccttgtcttttgggtgttgggaggatgagacattttctctgtatattttttattgtattaatttaaattatcataacaatttgaactattgattgctgtctactgatgctgaatatcaattctatttcatgacatatcagtagatttaaacaaatgcttagaatgtcctctcacattttgatttaatgtcttctacaatgccttctttcccccttgttctgtagggaatgtttcttaaaccaagtgaatatacttaaactttaacttgtactgttttttatctgcactagaatgatattagcttagtctgattaaacattaacttctgctgaaccccagcatttttgggataaggactacccaataaatgaaagagaggcaacttataacttgaactgtgcccactgcccagtggtggaaacatcaattgaacgcaaataacctttattcgtcattagccttcatcctcgtacactacctctatcaccatcacctataacttgaactgtttttcatctgccctagaatggcattaacttagtctgattaaacaaaaattctgcctaactccattgttttttggttctgtagcccatattccattcttttgtgaattgcgactattttcttttcttgtgaaagagtatggtacaactaaaagagaagctaaaagactaatctagggtagcctagggaatggtagagaaaaccctgcccatggagctatagaagatgtgccttccaataagagttaaatcatattcataagaggactctaattacaatagaatatcttgtatttgaaaaactattgtaaaaatgtgtgtggtacactgttacaaatagaatctaaaatagatatttttttcccttaaaaaaattgtagaagcacaattccgaaagattttatcttcacatataagccaccaggctgtcgactgcttccaaaaatgaccgtcaaccaagctagataggcaaaagataaaccatttttgttgaaaatgaaagaccatcctctacagaaaaaaggacagtggaggtaaaggtaattttgggattccccgttggaataacagaggaacaaaccaggggagagaacccagtttgggaaccttttttgagcttgtcttgggatgttgaggtcttcctaggcgtgtcaaacttccaaactgatttaaccgaaggcgttttgagctttagcaaggtctcagagagtttagtaaggtgattttagagaagagacaattgaatgaatcaatggtttctacctctatgtcttcgacagcttctaattgaactgtaacattctttcctatgaaggcaatccaactgacaatttctctatgaaaggagcaaacaagcactccacaattatcccccaaaattacctaaggatatggttttggtttcattgtctatttttctggtttgaccccatagatgccaaccatcacttttcataactatttaagatgtagggcattatttctctacttctgtaaatatccaatggaagggaagtccaattccattttaatgggtgccttaggatttggtgaattatgattatattgcattttgttgtagacaacaaccctctatacctcttttgttggaggactttagtttagattataagccatgttttaataggctaaaggcagtcctctggaagagacgaggtgtagcctcatggtgttatgaagcattaagtctcaaatttgattaatctttttttaccaagtattgctaaacaaaacttaataatcatgttaaagttaaaagaaaaaaagaaaaatcactcttcccaatttccacctggacaaaaattctaaaatgatatcttttacctcactactattatccgattgacttatcctagaaattttagactgtgacaaaataatattattcgacaaattattacaaagaggtgtaagcctcaataccttttgaaaacattggctataaataagtgtctctatatatgcaatatgcttgatgacctatattgcacttatgccttgtgaaatcttgattttgactgaagcatctaagttactttacctgtttcatctgtgttcgacaaattatttctaagacattctctagcgctacttagatgaatatcattgagcaacactacatcattgcatctgcaagtgtagatgtagtaattttcaatgatattacgttcattaattctttgtttcaattcaatgacactgcatcaacttaaagtagaagtatgtactctttttctcttaaaacctaagtattggtcgaggttagaagtcacatatggttgattttgagcggtttttcttgtattacttaggtgtggatatcagcatgaatacacatttgaaggctgtgaaacttactctaaaggggaaaaatccagttaccatggatcatttaagtgtgaggggtaacaacatcagatattatattctacccgacagcttgaatcttgagactttacttgttgaagagacacccagggtcaagcccaagaaaccaactgcaggtatgtcctgtccttgcattctggcatgtactttagtcaaattgtttatactgaatcatatgctatccattatcaaatgtgttgcttatgcatttctacactctactccaactacagggagacctttgggacgtggacgaggacgtggccgtgggcgtggtcgtgggcgtggacgctag

SEQ ID NO: 34 - DNA, 3913 bp, Solanum habrochaites
acgtttagtccaatggatgaggcagcccatgtgctaatgcgacgtcgttatagtgttttttgaataaccctggcgctctttgccctaaacctgaggtcgtgttcgatctgcgcttcgacttaagccctagtgtctcttcaatttcttcagcaacaatgaagctcgttaggttcatgcgatatcttcattttttgagtttcttttccagtgtgcttagcaatggatttaacttttaccttttttgtgcagatttttgatgaagctgaacaacgagactgtctcaattgagctcaaaaacggcaccgttgttcatggaaccattacaggtcaccctccctctctttctgtctatttctttattagctatatttaggtatttccctgtcaagtctaaaatgaacaatctgcacgtatgtaattgctgaattgctcgctccaataggtaaatagaatgtgcttgtataaaattcatttcctgggttgacaagttttttggtagtgtcgctaactgaaagtctagttgctgggtggtatatggaagttgctgaactacaacaacaatatactacccagtgtaattccacaagtggtgaggtcttgggagacagagtgtttgccttacccctacctcgtagtgtttaagtgactgtttccaatagattctcatgtttaaataaagcatttccaaaccatttttttaataaaggatatataaaaataagtgctcatgacagacaagtgaggttttgctcaagtggttgagcacttccaccgtcaactagtaggttgagggtttgggtcagaggatagatgggaacactattgatccttctaggctcctgggggtggggtggaaaaaacggagcaacaacaacaacgaaagcaagttgctaaactgtctttggtttatgccacaagttaagttagttcatagcgaatgttaactgcaatagtttacttcactctccttcacttccactgtgagtcactatgtatgtccataaatgcatggaagctaggactgagaggctatagaaagtatacgaggtaacccgtctcaatctgtctactcctctcactgcccttctgaatatgtctctgattcacacgtattatctctacttttgtcctccttcaactatgaagttcctttcaaataccaattggaattcattctcaagtgcagagctatttctgcctatcatcactgtaatttatgtctatactctaaccgtagttcctctcagttaatgcgttaattatactctgcttcaataagaataacatctctcgttcactttttgtggctctaaaaattggattattacacaactcgaagaattttgaagtgtcactataataattcaacaattccttcaatgaatttgtatgcatttaaataaactatgatgtctcaatcccaaactagttggtatgtctatattagtgtcctatatttgttctgctttatttggaccatttcattaattcacagttcgtatactgaaaatacaaatttctatattgaaaaacaaaagaaaaaaaaaagaaattatgggagtgaacatgggattcttatcatctttttgtcattttcttaatattttgtcataagttaaccccattgatatattctttgccttaaaacttaaactatgtttgactggggtggaagtggggcgtttgcgaaaggctatgtttagcgtgggggttggaagtggtagtgtttgcgaatgagtgtgtttagcgtgggggctgaagggtaagtcaggggtaaattagtaatttgacttttaagttaggagttgctttaaagtaatatagatagatatagataattactttatcctctgcaagaaaatcgtacagtagcttctttatctggcagtagagatgccttagcaaacttagataagcttcttatggcaagtgccagggcttgtgctggttgcatcgaaaatgtcaaattttgcaaggaaaatagccatatatgtattgcagagtttatctctattctagataactgtagtagtcaaaagtaaatttttgtcctgttctcatattttgagacctatagcatttctctttttctgttggttgtcctttccatgcttttgactgttattccttgaggggttgcttatacttacagaaaattggtagatatattcgggatatagaaatatgtatttgagcatatcagtatgtgttttgatattaactaggcacagcggctattaaatctgtctccttattgaagttgctagattgagagctgtgagttatagcttctaaaggcagttacatttttacaggtgtggatgttagcatgaacacacatctgaaggctgtcaaaattacgctaaaagggaagaatccagtgacgctggatcacctgagtgtgaggggtaacaacatccgttattacatcctccctgacagcttaaatcttgagacgctgctggtggaagaaacacctagggtgaagccaaagaagccaacagctggtattgtcatattgttaaggactatgttgggatttttttgtgccttgtgggtgtatggctaaatttccttattgcttggacatccttctaatatattcttcctttgattgttcctcttcaggaaagcctatgggacgtggtcgtgggcgcggtcgtgggcgcggacgtggtcgtggccgctaaatgcattccagctgctcttattttttgcagtgttgcctatgtaaacatgtgtacggatggagagatttgctcttagtttttcccagtagaaaaaagatggatctcatgcacaatttattttgtatctttgaagtcaggagcatgttatctcgttgcatcatattgtctgctaacattactttcgtatcgtgttcaattttgttctgccttgtcaagttactcaactaaattggtcactttagcatctatactctgaatagagtggcatagaattatatttggaccaaattgggaattgagataaatgatgatcggtgtggtataaggattgaaatccaggaatcgttgaaactcccatcaaattgggttttttaacttgtaaagtataaaacattcacaaatctttggaagtccagcaaaattgaagcctcggtattatttttgggttccaccgtcaaagttgtgaaattattccatgttttagtttttgtaacaacaagcgagtttttaattaaatgaaaatgagatactctatcagatgttgatatggctatttatgtaacttttcgatactcaaatcaatcgggcccattccttctatcttaattcctcttctggtatataaaaatagagaagcaaatcatctcgagagctgcaaacgaaaaccctcatcggcacaaggtgcttgtggtctctttcaatggcgaagaataactcgttgaagtcgaccgcagtggtattcggagccctagcatttgggtggctggcgattgagcttgctttcaaaccatggcttgataaagcccgagcttccatggacaagtctgacccatctcgcgaccccgacgatcaaagttcagaaactggcaagtctcaggtcgatgttgatccgaacaactgatttgatttttgggttctaaaccccaaaatccaaatatatgtgcttgaattttacttcataataaataattatgacttgttaatttctgtttttcctcagtttctagccgtccctgtattttattttgttggttaaagtgaaagaaaaaaaaaagaattggtagaagtttaatacaattcaagaggtaagttcaagataattgttccgtttttatgtttctgtccatttgtatttgtataaaatgatattttgacatttaacgtgttgctgttgatttatcatataaaaatttgtctacacaaatgagaatgagaaagagggc

SEQ ID NO: 35 - DNA, 4290 bp, Solanum pennellii
taaaagtaggtgatttttataattttacatacatagatacctaagaggtgctattattcttatattcttagcattaaatagtgtgagacgcggcccacgtttagtccaatggacgttcccagacgaggcgtataataagacatcacttgtgttttttaaacaacccctggcgctccttgccctaaactaaagccctagattagtgtgttttcaatttcttcagcaagcaacaatgaagctcgtcaggttcatgcgatatcttcattttttttgagtttcttttccagtgagcttagcaatggattttaactttacttttttgtgcagatttttgatgaagctgaacaacgagactgtctcaattgagctcaaaaacggcaccgttgttcatggaaccattacaggtcaccctccttatctttctttctttgctaaattgaacaatgtaatttctcaagccaatatgtgaattaaataatgtgtttgtatgaatttcaagttatttggaagtgtcgctaactgaactacaacaacaacataataattccacaagtggtgaggtcgtagagttaaagaggttctttccaatagattttcatgcttaaataaagcatttccaaacaatttttgtaaaggatatacaaaaattagtgctcatgacagacaagtgaggttttgctcaagtggttgagcactggtaggttgagggtttgggtcagaggatagatcggaacactattgatccttctaagctcctaggggtggggtggaaaaacggagcagcaacaacaacgaaaccaagttgctaaactatctttggtttatgccacgagttaagttagtttggtaactgcaatagtttacttcactctccttcacttccgctgtgagtcactatgtatgtccatgaatgcatcgaagctagggctgagaagctatagaaagtatatgtggtaactcgtctcaatctgtctactcctctcactgcccttctgaatttgttatcttcttatcctctctgattcacacgtattatctctacttcagtccttcaactatgaagttcctttcaaataccaataggaattcattctcaagtgcagagctatttctacctatcatcactgtaatttatgtctagactctagatcactgtagttcatctctgtcaatgcatttactgtactctgggtcaataaaaataacatcactcgttccctttttttgtagctctaaaagttggattattacacaactcgaagaaatttgaagtgtcactataataattagtatgcatttaaatacaactgtgatgtctcaatcccaaactagttggtttgtctatatgagtgttctctattttttctgctttattcggaccatttcattaattcacagttcgtatactgaaaatacaaatctctaaattaaaaaacaaaacaaaaaactaagaaattatgggagtgaacatgggattcttatcatctttttgtcattttcttaatatttctcttaaattaaccccattcatatattctttgctttgaaacttaaactatgtttggctggggtggaaaaaagaagcttaagaggaatggaagtaagaggttaagagttaatttcgttctaagtaaaaaagggatataaagcaaatgtagatgggttgtgtatccttctatgtggatttttcagtttatctgaattttgatggaaaaaggaaatgtatttgtcttatttctgttccagccataccaaacaacctgaactacatcgcttctatacacataagggaggaaaatcactgaataaccaatacaccacataacgggaaagtaaatcagtgaccaatgcttcacttaagggaggtagatcagaagaatctgtcttgacttcatattgagatagtagaatttgtaatacttctaaattttcacgggtaactgtcaccattaatttgatacatcagtagaaagtataaagaatttttggtacttttatactgctaatattttcaatgttgtgtaatattagcctgagataagtttaaacagagtaacatggtcaatgggattcatttagtcggaccgaacatttttgaaactgtggtgttgtttctttcgttgatgttgtaagaccttcattatcattttgtcaatagaataggtcgataatgtaatgatcttcagaaatttaagtctgcacatggttccacatcattatgccaccattgttctcaggtccctacaagtgaaacattctatgctggaatcatttggtccaagagctcttttattttctatgcaaaaccatgttgtctttatccttttgtttccaaagatgtgcattagttgctcatctcagcaaatgtaacactaattgacaaaacctacgttcaatattaaattttatctgtataaaattttgattacctagttgttggtatacgggtaacttataacacttgttagttgattatatgagcaaaaaccatgttgcctccacttgtttgtggattttcattgtctatgtgaggcgtgagtaagttcttatttcccagctcgagcttaagttccaggtacttaggtcagaaagagtacgacacgtcaagttctataggctggttgcatttgatatttgagcatgtagaactagcgcaaagagtgagaagtaagtggctatatgatgttgtgtactctgacttttgctgtgcaatgttaactaatcttaaattcaactagatgaggagctaaagttgtgtcgaacaactattattcactatgcatgaatacttgagcttatgcgagcgtgcgatcaacttatagcttattatgttgtacccaagttttatgaaaacaacacctagtgttgcgtttataattcttatccgttgttgtaatcttgttgtgaatgttctatgtaattcattgtactatagattttttaccattgaggagtagccatttatcccttatttctttttggtgctcccatgatatacctctatgcattgaagataagtgtcattcatagaatgatcgaacatcatatctttaggtaggcgttctgctttttggtatacttatacaggcgtgctttgtgcctacatttaaaaaaataataattgctttatcctctaaaaaagatcatcatactgtagcttctttgtctggctgtagagatgccttagcaaacttagatcggcttcttatggcaagtgccagggcttgtgctggttgcatcgaaaatgtcaaatttgcatggaaaatagccactatgtattgcagagtttatctctgttctagataactttagtaggcagaagtagatattttgtcctgttctcatattttgagacctatagcattttccatgcttttgacttttattccttgaggggttgcttatacttgcagaaaattgctagatatcagtatgtgttttgatattaactaggcacatcggctattacatctgtctccttattgaagttgctaaattgagagctgtgagttaaagtttctaaaggcagttacatttttacaggtgtggatgttagcatgaacacacatctgaaggctgtcaaaattacgctaaaagggaagaatccagtgacgttggatcacctgagtgtgaggggtaacaacatccgttattacatcctccctgacagcttaaatcttgagacgttactggtggaagaaacacctagggtgaagccaaagaagccaacagctggtattgttaaggactacgttcagatgtttttgtgcattgtgggtgtatggttaaattgcctttttgcttggacatccttctaatacgctcttgctttgattgttcctcttcaggaaagcctatgggacgtggtcgtgggcgcggtcgtgggcgtggacgtggtcgaggccgctaaatgcattccagctggtctatttttttgcagtcttgcctatgtaaacatgtgtacggattgatagatttgctcttagtttttatcctgtagaaaaaagatggatctcatgcaacaatttattttggatctttgaagtcaggagcatgttatctcgttgcatcatattgtctgctaacattaatttcgtttcgcgttcaatttagtcaccttatgcatgttttgagagttgagtcacatttaatgtcgattgagaattcagctccttcttatgaattatacttcttataggaaatagccttctttggtgtgactcggagggggaatattgttcctcttacttccccttttagtctgtttcaaaaaaaatgtcactttcttct